(19) United States
(12) Patent Application Publication
(10) Pub. No.: US 2026/0308675 A1
(43) Pub. Date: Oct. 8, 2026

(54) IMPROVED RNA IN VITRO TRANSCRIPTION USING DNA BEADS

(71) Applicant: CureVac Manufacturing GmbH, Tübingen (DE)

(72) Inventors: Michael THOMMEN, Tübingen (DE); Hiba AL ABDALLAH, Tübingen (DE); Sina KLEIN, Tübingen (DE); Elisa HIRTE, Tübingen (DE); Lena EGGERS, Tübingen (DE); Tilmann ROOS, Tübingen (DE); Felix BERTSCH, Tübingen (DE); Benyamin YAZDAN PANAH, Tübingen (DE)

(73) Assignee: CureVac Manufacturing GmbH, Tübingen (DE)

(21) Appl. No.: 19/484,340

(22) PCT Filed: May 16, 2023
(86) PCT No.: PCT/EP2023/063072

§ 371 Date: Nov. 13, 2025

(51) Int. Cl. C12Q 1/6865; C12P 19/34

(52) U.S. Cl. CPC C12Q 1/6865; C12P 19/34

(57) ABSTRACT

The present invention is inter alia directed to RNA in vitro transcription compositions comprising DNA templates immobilized on beads (DNA beads) that are contained in an RNA in vitro transcription (IVT) buffer, wherein said composition is configured to reduce or prevent an agglomeration of the DNA beads during the course of RNA in vitro transcription. The problem of DNA beads agglomeration has not yet been recognized in the art and can be solved according to the invention by optimizing the components of the IVT buffer (e.g. adjusted concentration of spermidine and/or Mg²⁺). Further aspects inter alia relate to a method of producing RNA using the RNA in vitro transcription composition, an RNA manufacturing device.

### INTRODUCTION

The present invention is inter alia directed to RNA in vitro transcription compositions comprising DNA templates immobilized on beads (also referred to as DNA beads) that are contained in an RNA in vitro transcription (IVT) buffer, wherein said composition is configured to reduce or prevent an agglomeration of the DNA beads for example during the course of RNA in vitro transcription. The problem of DNA beads agglomeration has not yet been recognized in the art and can be solved according to the invention by optimizing the components of the IVT buffer. Further aspects relate to a method of producing RNA using the RNA in vitro transcription composition, an RNA manufacturing device, and several uses.

Therapeutic RNA represents an emerging class of drugs. RNA-based therapeutics include mRNA molecules encoding antigens for use as vaccines. In addition, it is envisioned in the field to use RNA molecules for protein replacement therapies or cancer therapies. Furthermore, the therapeutic use of noncoding immunostimulatory RNA molecules, other noncoding RNAs such as microRNAs and long noncoding RNAs, or RNAs suitable for genome editing (e.g., CRISPR/Cas9 guide RNAs) is explored. Accordingly, RNA-based therapeutics belong to the most promising and quickly developing therapeutic fields in modern medicine.

Established manufacturing processes for RNA molecules implement many separate manufacturing steps. One critical step in RNA production is the generation of a suitable DNA template, which at industrial scale is a major cost factor. Typically, DNA templates can only be used for a single RNA in vitro transcription reaction and need subsequently be destroyed by DNase digestion and eventually removed by purification to ensure efficacy and safety of the RNA-based therapeutics. In consequence, established manufacturing processes are time consuming, cost intensive, and require a lot of laboratory space and laboratory equipment.

Currently emerging RNA manufacturing processes make use of immobilized DNA templates. For example, the immobilization of PCR-amplified DNA templates (e.g., WO2020002598) or linearized plasmid DNA templates (e.g., WO2019122371) on beads has been recently described. DNA immobilization has the advantage that the generated DNA-beads can be re-used for several RNA in vitro transcription cycles, that DNA beads can be used in automated production processes, and that no DNase digestion steps is needed as DNA-beads can be separated from the obtained RNA product by e.g., magnetic separation or filtration.

However, RNA in vitro transcription using DNA beads, in particular an automated RNA in vitro transcription using DNA beads, is not well established in the field and may therefore require further optimizations. Accordingly, an object of the present invention is to provide optimized RNA in vitro transcription compositions and optimized methods for RNA synthesis based on DNA templates immobilized on beads.

These optimized compositions and methods are particularly suitable for the pharmaceutical production of RNA, in particular, for the automated production of therapeutic RNA.

### SUMMARY OF THE INVENTION

RNA in vitro transcription using DNA immobilized on beads is not well established in the field and may therefore be associated with several problems.

As outlined in the Examples section, the inventors identified an as yet undescribed problem relating to an unwanted DNA bead agglomeration that can occur during the course of RNA in vitro transcription (IVT), in particular after more than one IVT cycle. Consequently, DNA bead agglomeration may inter alia reduce the yield of the produced RNA, in particular if more than one IVT cycle is carried out, may interfere with automated processing steps, and may lead to a contamination of the final RNA product. In extensive studies described in the Examples section, the inventors found that certain parameters of the RNA in vitro transcription buffer (IVT buffer) and/or the DNA beads may be adapted to reduce or prevent an unwanted DNA bead agglomeration which has a strong negative impact on RNA yield. Moreover, the inventors developed RNA in vitro transcription methods using DNA beads that are particularly suitable to reduce or prevent DNA bead agglomeration.

Accordingly, in a first aspect, the present invention provides an RNA in vitro transcription composition comprising DNA templates immobilized on beads (herein referred to as DNA beads) that are contained in an RNA in vitro transcription (IVT) buffer.

Suitably, the RNA in vitro transcription composition is configured to reduce or prevent an agglomeration of the DNA beads during the course of RNA in vitro transcription.

The RNA in vitro transcription composition comprises DNA templates immobilized on beads, wherein the beads are contained in an RNA in vitro transcription (IVT) buffer that may comprise
less than 2 mM spermidine; and/or
Mg²⁺ to NTP in a molar ratio of below 1.4.

The IVT buffer may be characterized by at least one or a combination of the following features F1 to F6
F1: less than 1 mM spermidine, preferably free of spermidine;
F2: Mg²⁺ at a concentration between 1 mM to 25 mM, preferably less than 20 mM Mg²⁺;
F3: the added total concentration of spermidine and Mg²⁺ in the IVT buffer is less than 25 mM;
F4: the molar ratio of Mg²⁺ to NTPs is ranging from 1.2 to 0.5, preferably ranging from 0.8 to 0.6;
F5: NTPs at a total concentration of at least 3 mM, preferably ranging from 3 mM to 35 mM;
F6: less than 10 mM DTT, preferably less than or about 1 mM DTT;

The DNA beads may be characterized by at least one or a combination of the following features B1 to B5
B1: comprise a magnetizable material, preferably a (super) paramagnetic material;
B2: diameter in a range of 1 μm to 10 μm, in a range of 4 μm to 5 μm, e.g. 4.5 μg;
B3: comprise polystyrene or a derivative of polystyrene;
B4: the bead surface loading comprises about 1 ng DNA/mm² to about 5 ng DNA/mm²
B5: beads comprise a biotin-streptavidin immobilized DNA template;

In a second aspect, the present invention provides the use of the RNA in vitro transcription composition of the first aspect in a manufacturing process of RNA.

In a third aspect, the present invention provides a method for producing RNA using an RNA in vitro transcription composition comprising DNA templates immobilized on beads that are contained in an RNA in vitro transcription (IVT) buffer.

Suitably, the RNA in vitro transcription composition is further characterized by any one of the features to the first aspect.

A fourth aspect of the present invention relates to an RNA manufacturing device that comprises the RNA in vitro transcription composition of the first aspect or that is configured to carry out the method of the third aspect.

Preferably the device comprises a magnet unit for mixing the DNA beads and/or for retaining the DNA beads.

A fifth aspect of the present invention relates to the use of an IVT buffer to prevent or reduce agglomeration of DNA beads. Suitably, the IVT buffer of the use is characterized according to the first aspect.

A further aspect of the present invention relates to the use of a buffer comprising a chelating agent to prevent or reduce agglomeration of DNA beads during the course of RNA in vitro transcription.

### Definitions

For the sake of clarity and readability the following definitions are provided. Any technical feature mentioned for these definitions may be read on each and every embodiment of the invention. Additional definitions and explanations may be specifically provided in the context of these embodiments.

About: The term “about” is used when determinants or values do not need to be identical, i.e., 100% the same. In the context of the invention, “about” means, that a determinant or values may diverge by no more than 20%, preferably by no more than 10%, in particular by no more than 5%.

Agglomeration: The term “agglomeration” as used herein in the context of beads, in particular DNA-beads, relates to assemblies of beads that collectively aggregate or agglomerate. Agglomeration is when beads are combined loosely or when beads coherently attach. Typically, such agglomerates can be broken by mechanical forces. Agglomeration as used herein has to be distinguished from capturing of beads by means of a magnet which can cause a temporal accumulation of beads near the magnet. However, after loosening the magnetic force, the temporal accumulation of beads resolves, and the beads disperse in a composition.

Coding sequence/coding region: The terms “coding sequence” or “coding region” and the abbreviation “cds” as used herein will be recognized and understood by the person of ordinary skill in the art, and are e.g., intended to refer to a sequence of several nucleotide triplets that may be translated into a peptide or protein. A cds in the context of the present invention may be an RNA sequence consisting of a number of nucleotides that may be divided by three, which starts with a start codon and preferably terminates with a stop codon.

Heterologous: The terms “heterologous” or “heterologous sequence” as used throughout the present specification in the context of an RNA sequence or an amino acid sequence refers to a sequence (e.g., RNA, amino acid) has to be understood as a sequence that is derived from another gene, another allele, or e.g., another species or virus. Two sequences are typically understood to be “heterologous” if they are not derivable from the same gene or from the same allele. I.e., although heterologous sequences may be derivable from the same organism or virus, in nature, they do not occur in the same nucleic acid or protein.

RNA: The terms “RNA” and “mRNA” are e.g., intended to be a ribonucleic acid molecule, i.e., a polymer consisting of nucleotides. These nucleotides are usually adenosine-monophosphate, uridine-monophosphate, guanosine-monophosphate and cytidine-monophosphate monomers which are connected to each other along a so-called backbone. The backbone is formed by phosphodiester bonds between the sugar, i.e., ribose, of a first and a phosphate moiety of a second, adjacent monomer. The specific succession of the monomers is called the RNA-sequence. The mRNA (messenger RNA) provides the nucleotide coding sequence that may be translated into an amino-acid sequence of a particular peptide or protein.

RNA in vitro transcription: The term “RNA in vitro transcription” relates to a process wherein RNA is synthesized in a cell-free system. RNA may be obtained by DNA-dependent RNA in vitro transcription of an appropriate DNA template, which according to the present invention may be a DNA template immobilized on beads. The promoter for controlling RNA in vitro transcription can be any promoter for any DNA-dependent RNA polymerase. Particular examples of DNA-dependent RNA polymerases are the T7, T3, SP6, or Syn5 RNA polymerases. RNA in vitro transcription takes place in an in vitro transcription buffer (IVT buffer) that comprises the components required to transcribe the DNA template into RNA. A typical IVT buffer may comprise: ribonucleotide triphosphates (NTPs) (e.g. adenine, cytosine, guanine and uracil); optionally, a cap analogue (e.g. a Cap1 analog); optionally, further modified nucleotides (e.g. N1-methylpseudouridine (m1 w)); a DNA-dependent RNA polymerase capable of binding to the promoter sequence within the DNA template (e.g. T7, T3, SP6, or Syn5 RNA polymerase); optionally, a ribonuclease (RNase) inhibitor to inactivate any potentially contaminating RNase; optionally, a pyrophosphatase to degrade pyrophosphate (inhibitor of RNA synthesis); MgCl₂, which supplies Mg²⁺ ions as a co-factor for the polymerase; a buffer substance (e.g. TRIS or HEPES) which can also contain antioxidants (e.g. DTT); optionally, polyamines such as spermidine.

### DETAILED DESCRIPTION OF THE INVENTION

Where reference is made to “SEQ ID NOs” of other patent applications or patents, said sequences, e.g., amino acid sequences or nucleic acid sequences, are explicitly incorporated herein by reference. Where reference is made to “SEQ ID NOs” in the context of RNA sequences, the skilled person will be able to derive RNA sequences from the referenced SEQ ID NOs also in cases where DNA sequences are provided. Where reference is made to “SEQ ID NOs” in the context of DNA sequences, the skilled person will be able to derive respective DNA sequences from the referenced SEQ ID NOs also in cases where RNA sequences are provided.

### RNA In Vitro Transcription Composition:

In a first aspect, the present invention provides an RNA in vitro transcription composition.

Preferably, the RNA in vitro transcription composition comprises DNA templates immobilized on beads, wherein said beads are contained in an RNA in vitro transcription (IVT) buffer.

It has to be noted that features and embodiments described and defined in the context of the first aspect, in particular features and embodiments relating to the IVT buffer or the DNA beads, may likewise be applicable to any of the other aspects of the present invention.

In the context of the invention, beads that comprise immobilized DNA templates as specified herein are also referred to as DNA beads. Suitably, said DNA beads are contained in the IVT buffer as dispersed DNA beads or particles. Preferably, induced by a mixing or swirling of the RNA in vitro transcription composition (e.g. via a magnetic force or a shaker), the DNA beads are dispersed in the IVT buffer as free-floating DNA beads.

As described herein, the inventors encountered an as yet undescribed problem of DNA bead agglomeration that occurs when carrying out an RNA in vitro transcription reaction using DNA beads as a template. The IVT buffer and the DNA beads have therefore been adapted to reduce or prevent an agglomeration of DNA beads. Bead agglomeration can be determined using microscopy at a 500 fold or a 1000 fold magnification.

In preferred embodiments, the RNA in vitro transcription buffer is configured to reduce or prevent agglomeration of the DNA beads e.g. during the course of RNA in vitro transcription. In preferred embodiments, the RNA in vitro transcription buffer is configured to reduce or prevent agglomeration of the DNA beads (e.g., beads are dispersed and/or free floating) in a first, second, and/or a third RNA in vitro transcription reaction (e.g. an IVT cycle).

In preferred embodiments, the RNA in vitro transcription composition comprises DNA templates immobilized on beads contained in an RNA in vitro transcription (IVT) buffer, wherein the IVT buffer comprises
less than 2 mM spermidine; and/or
less than 25 mM Mg2+; and/or.
Mg2+ and NTP in a molar ratio of below 1.4.
In preferred embodiments, the RNA in vitro transcription composition comprises DNA templates immobilized on beads contained in an RNA in vitro transcription (IVT) buffer, wherein the IVT buffer comprises
less than 2 mM spermidine; and
Mg2+ and NTP in a molar ratio of below 1.4.

In preferred embodiments, the RNA in vitro transcription composition comprises DNA templates immobilized on beads contained in an RNA in vitro transcription (IVT) buffer, wherein the IVT buffer comprises
less than 25 mM Mg2+; and
Mg2+ and NTP in a molar ratio of below 1.4.

In particularly preferred embodiments, the RNA in vitro transcription composition comprises DNA templates immobilized on beads contained in an RNA in vitro transcription (IVT) buffer, wherein the IVT buffer comprises
less than 2 mM spermidine; and
less than 25 mM Mg2+; and
Mg²⁺ and NTP in a molar ratio of below 1.4.

In the following, preferred features of the IVT buffer are further specified.

### Polyamines Such as Spermidine

Typically, IVT buffers comprise polyamine compounds such as spermidine. Spermidine is usually considered to be a critical component for RNA in vitro transcription, inter alia because spermidine is thought to improve the overall efficiency of RNA polymerases. However, as described herein, polyamines such as spermidine may cause or promote an unwanted agglomeration of the DNA beads, an unexpected an as yet undescribed negative effect which can e.g., lead to a reduction of RNA yield (in particular if more than one IVT cycle is carried out), and/or RNA quality. Therefore, it is preferred to reduce the amount of polyamine compounds such as spermidine in the IVT buffer.

In preferred embodiments, the IVT buffer comprises spermidine at a concentration of less than 2 mM, less than 1.5 mM, less than 1.0 mM, preferably less than 500 μM. In particularly preferred embodiments, the IVT buffer is essentially free of spermidine.

In preferred embodiments, the IVT buffer comprises a polyamine compound at a concentration of less than 2 mM, less than 1.5 mM, less than 1 mM, preferably less than 500 μM, more preferably the IVT buffer is essentially free of polyamine compounds.

Surprisingly, the performance of RNA in vitro transcription (e.g., RNA yield) using DNA beads is not impaired by a reduced spermidine or polyamine concentration in the IVT buffer (e.g. less than 2 mM). Even more surprisingly, the performance of RNA in vitro transcription using DNA beads is not impaired if the reaction is performed in an IVT buffer that is essentially free of spermidine or an alternative polyamine compound.

### Mg2+ Ions

Typically, IVT buffers comprise divalent cations, usually Mg²⁺, that are needed as co-factors for the RNA polymerases. The Mg²⁺ ions are typically provided by magnesium salts such as MgCl₂ or MgOAc₂. However, as described herein, divalent cations such as Mg²⁺ may cause or promote an unwanted agglomeration of the DNA beads, an unexpected an as yet undescribed negative effect which can e.g., cause a reduction of RNA yield and/or RNA quality. In preferred embodiments in that context, Mg²⁺ is provided by MgCl₂. In preferred embodiments, Mg²⁺ is kept as low as possible to minimize the risk of DNA bead agglomeration.

In preferred embodiments, the IVT buffer comprises less than 30 mM divalent cations, preferably less than 25 mM divalent cations or less than 20 mM divalent cations.

In preferred embodiments, the IVT buffer comprises Mg²⁺ at a concentration of less than 25 mM, more preferably less than 20 mM. In embodiments, the concentration of Mg²⁺ can be further reduced in particular in embodiments where NTPs are fed during the course of RNA in vitro transcription as further described in the context of the third aspect. Accordingly, the IVT buffer can comprise Mg²⁺ at a concentration of less than 20 mM, less than 15 mM, less than 10 mM, or less than 5 mM.

In preferred embodiments, the IVT buffer comprises Mg²⁺ at a concentration between 1 mM to 25 mM, between 2 mM to 25 mM, preferably ranging from 5 mM to 20 mM, more preferably ranging from 10 mM to 20 mM or 15 mM to 20 mM. In particular, the IVT buffer comprises Mg²⁺ at a concentration ranging from 18 mM to 20 mM. In specific embodiments, the IVT buffer comprises about 18 mM Mg²⁺, about 19 mM Mg²⁺, or about 20 mM Mg²⁺.

In embodiments, the concentration of Mg²⁺ can be further reduced in particular in embodiments where NTPs are fed during the course of RNA in vitro transcription as further described in the context of the third aspect. Accordingly, the IVT buffer can comprise Mg²⁺ at a concentration ranging from 1 mM to 20 mM, preferably ranging from 1 mM to 15 mM. In particular, the IVT buffer comprises Mg²⁺ at a concentration ranging from 2 mM to 15 mM, 2 mM to 10 mM, 2 mM to 5 mM. In specific embodiments, the IVT buffer comprises about 2 mM Mg²⁺, about 3 mM Mg²⁺, or about 4 mM Mg²⁺.

Surprisingly, the performance of RNA in vitro transcription (e.g., RNA yield) using DNA beads is not impaired by a reduced Mg²⁺ concentration (e.g., less than 25 mM Mg²⁺) in the IVT buffer.

### Divalent or Trivalent Ions

Typically, IVT buffers comprise cationic components (e.g., Mg²⁺ ions and/or polyamine compounds such as spermidine). However, as described herein, these cations may cause or promote an unwanted agglomeration of the DNA beads, an unexpected an as yet undescribed negative effect which can e.g., cause a reduction of RNA yield (in particular if more than one IVT cycle is carried out), and/or RNA quality. Therefore, it is preferred to reduce the overall amount of cations in the IVT buffer.

In embodiments, the IVT buffer comprises less than 30 mM divalent or trivalent cations, less than 25 mM divalent or trivalent cations, preferably less than 20 mM divalent or trivalent cations. In embodiments, the IVT buffer can comprise less than 15 mM, less than 10 mM, less than 5 mM divalent or trivalent cations.

In preferred embodiments, the added total concentration of Mg²⁺ and spermidine in the IVT buffer is less than 30 mM, preferably less than 25 mM, more preferably less than 20 mM. In embodiments, the concentration of Mg²⁺ and spermidine can be further reduced in particular in embodiments where NTPs are fed during the course of RNA in vitro transcription. Accordingly, the IVT buffer can comprise Mg²⁺ and spermidine at an added total concentration of less than 20 mM, less than 15 mM, less than 10 mM, or less than 5 mM.

In preferred embodiments, the IVT buffer comprises Mg²⁺ and spermidine at an added total concentration ranging from 1 mM to 30 mM, ranging from 2 mM to 25 mM, preferably ranging from 5 mM to 20 mM, more preferably ranging from 10 mM to 20 mM or 15 mM to 20 mM. In particular, the IVT buffer comprises Mg²⁺ and spermidine at an added total concentration ranging from 18 mM to 20 mM. In specific embodiments, the IVT buffer comprises about 18 mM Mg²⁺ and spermidine, about 19 mM Mg²⁺ and spermidine, or about 20 mM Mg²⁺ and spermidine.

In embodiments, the added total concentration of Mg²⁺ and spermidine can be further reduced in particular in embodiments where NTPs are fed during the course of RNA in vitro transcription. Accordingly, the IVT buffer can comprise Mg²⁺ and spermidine at an added total concentration ranging from 1 mM to 20 mM, preferably ranging from 1 mM to 15 mM. In particular, the IVT buffer comprises Mg²⁺ and spermidine at an added total concentration ranging from 2 mM to 15 mM, 2 mM to 10 mM, 2 mM to 5 mM. In specific embodiments, the IVT buffer comprises about 2 mM Mg²⁺ and spermidine, about 3 mM Mg²⁺ and spermidine, or about 4 mM Mg²⁺ and spermidine.

### NTPs

IVT buffers typically comprise a ribonucleotide triphosphates (NTPs) mixture. To allow efficient RNA in vitro transcription without causing agglomeration of the DNA beads, the amount of NTPs may be adjusted to an appropriate value.

In preferred embodiments, the NTP mixture comprises adenine, cytosine, guanine and uracil.

In preferred embodiments, the NTP mixture comprises modified nucleotides.

In preferred embodiments, the modified nucleotide of the NTP mixture is a modified uracil nucleotide.

In some embodiments, the modified nucleotide is selected from pseudouridine, N1-methylpseudouridine, N1-ethylpseudouridine, 2-thiouridine, 4-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. Accordingly, in preferred embodiments, the IVT buffer of the invention comprises a modified nucleotide selected from pseudouridine (ψ) and/or N1-methylpseudouridine (m1ψ). Particularly preferred in the context of the invention is N1-methylpseudouridine (m1ψ).

In preferred embodiments in that context, the IVT buffer comprises an NTP mixture that contains G, C, A and U nucleotides as defined herein. Accordingly, in that embodiment, the IVT buffer does not comprise modified nucleotides.

In other preferred embodiments in that context, the IVT buffer comprises an NTP mixture that contains G, C, A and m1ψ as defined herein.

In alternative embodiments in that context, the IVT buffer comprises an NTP mixture that contains G, C, A and ψ as defined herein.

In preferred embodiments, the NTPs mixture is optimized for the given RNA sequence to be produced (according to claims 1 to 35 of WO2015188933). Accordingly, for producing an RNA sequence with G:C:A:U of 1:2:3:2, the respective sequence optimized NTP mixture comprises G:C:A:U in a molar ratio of 1:2:3:2.

In preferred embodiments, the IVT buffer comprises NTPs at a total concentration of at least 1 mM, at least 2 mM, at least 3 mM.

In preferred embodiments, the IVT buffer comprises NTPs at a total concentration ranging from 1 mM to 45 mM, ranging from 1 mM to 35 mM, ranging from 3 mM to 35 mM, preferably ranging from 7 mM to 30 mM, more preferably ranging from 15 mM to 30 mM or ranging from 20 mM to 30 mM. In particularly preferred embodiments, the IVT buffer comprises NTPs at a total concentration ranging from 26 mM to 28 mM. In a specific embodiments, the IVT buffer comprises about 26 mM NTPs, about 27 mM NTPs, about 28 mM NTPs.

In embodiments, the concentration of NTPs can be further reduced in particular in embodiments where NTPs are fed during the course of RNA in vitro transcription. Accordingly, the IVT buffer can comprise NTPs at a concentration ranging from 2 mM to 30 mM, preferably ranging from 2 mM to 20 mM. In particular, the IVT buffer can comprise NTPs at a concentration ranging from 3 mM to 20 mM, 3 mM to 15 mM, 3 mM to 7 mM. In specific embodiments, the IVT buffer comprises about 3 mM NTPs, about 4 mM NTPs, or about 5 mM NTPs.

Notably, the term “total concentration” of the NTPs (the NTP mixture) relates to the added molar concentration of ribonucleotides (e.g., A, G, U, C) and modified ribonucleotides (e.g., m1ψ, ψ) that may be comprised in the NTPs (the NTP mixture) of the IVT buffer.

In preferred embodiments in that context, the NTP (the NTP mixture) may comprise modified nucleotides as defined herein, preferably selected from pseudouridine (w) or N1-methylpseudouridine (m1ψ). Most preferably, the NTPs comprise N1-methylpseudouridine (m1ψ).

In a particularly preferred embodiment, the IVT buffer comprises NTPs at a total concentration as defined herein, wherein the NTPs comprise N1-methylpseudouridine. In a specific embodiment, the IVT buffer comprises about 27 mM NTPs, wherein the NTPs comprise N1-methylpseudouridine.

### NTP to Mg2+ Ratio

As described above, IVT buffers comprise NTPs and Mg²⁺ cations to facilitate RNA synthesis. To allow efficient RNA in vitro transcription without causing agglomeration of the DNA beads, the ratio (e.g., molar ratio) of Mg²⁺ to NTPs may be adjusted to an appropriate value. In state-of-the-art IVT protocols, the Mg²⁺ to NTPs ratio is typically larger than 1.5.

In preferred embodiments, the molar ratio of Mg²⁺ to NTPs (the total NTP concentration as defined herein) in the IVT buffer is below 1.4, preferably below 1.2, more preferably below 1.0. For example, the molar ratio of Mg²⁺ to NTPs is at about 1.3, 1.2, 1.1, 1.0, 0.9, 0.8, 0.7, 0.6, 0.5, or 0.4.

In preferred embodiments, the molar ratio of Mg²⁺ to NTPs (the total NTP concentration as defined herein) in the IVT buffer is ranging from 1.4 to 0.5, 1.3 to 0.5, 1.2 to 0.5, 1.1 to 0.5, preferably ranging from 1 to 0.5, more preferably ranging from 0.8 to 0.6. In specific embodiments, the molar ratio of Mg²⁺ to NTPs (the total NTP concentration as defined herein) is at about 0.8, about 0.7, or about 0.6. In particularly preferred embodiments, the molar ratio of Mg²⁺ to NTPs (the total NTP concentration as defined herein) in the IVT buffer is at about 0.7.

Surprisingly, the performance of RNA in vitro transcription (e.g., RNA yield) using DNA beads is not impaired by a Mg²⁺ to NTPs ratio that is below a value of 1.4. Even more surprisingly, the performance of RNA in vitro transcription using DNA beads is not impaired if the ratio of Mg²⁺ to NTP is below 1.2. Reducing the Mg²⁺ to NTP ratio in the RNA in vitro transcription has the advantageous effect that DNA bead agglomeration can thereby be reduced and/or prevented.

In preferred embodiments, the molar ratio of Mg²⁺ to NTPs in the IVT buffer is ranging from 1.5 to 0.5, 1.2 to 0.5, preferably ranging from 1 to 0.5, more preferably ranging from 0.8 to 0.6. (e.g. 0.7) and the IVT buffer comprises Mg²⁺ at a concentration between 1 mM to 25 mM, between 2 mM to 25 mM, preferably ranging from 5 mM to 20 mM, more preferably ranging from 10 mM to 25 mM or 15 mM to 20 mM, in particular ranging from 18 mM to 20 mM. In specific embodiments in that context the molar ratio of Mg²⁺ to NTPs in the IVT buffer is ranging from 1.5 to 0.5, 1.2 to 0.5, preferably ranging from 1 to 0.5, more preferably ranging from 0.8 to 0.6. (e.g. 0.7) and the IVT buffer comprises about 18 mM Mg2+, about 19 mM Mg2+, or about 20 mM Mg²⁺.

In other embodiments', the molar ratio of Mg²⁺ to NTPs in the IVT buffer is ranging from 1.5 to 0.5, 1.2 to 0.5, preferably ranging from 1 to 0.5, more preferably ranging from 0.8 to 0.6. (e.g. 0.7) and the IVT buffer comprises Mg²⁺ at a concentration ranging from 1 mM to 20 mM, preferably ranging from 1 mM to 15 mM, in ranging from 2 mM to 15 mM, 2 mM to 10 mM, 2 mM to 5 mM. In specific embodiments in the context the molar ratio of Mg²⁺ to NTPs in the IVT buffer is ranging from 1.5 to 0.5, 1.2 to 0.5, preferably ranging from 1 to 0.5, more preferably ranging from 0.8 to 0.6. (e.g. 0.7) and the IVT buffer comprises about 2 mM Mg2+, about 3 mM Mg²⁺, or about 4 mM Mg²⁺.

In preferred embodiments in that context, the molar ratio of Mg²⁺ to NTPs (the total NTP concentration as defined herein) in the IVT buffer is ranging from 0.8 to 0.6 and the respective Mg²⁺ and NTP concentrations are selected from:
Mg²⁺ at a concentration of 1 mM to 6 mM and NTPs at a concentration of 1.3 mM to 10 mM;
Mg²⁺ at a concentration of 3 mM to 9 mM and NTPs at a concentration of 3.8 mM to 15 mM;
Mg²⁺ at a concentration of 6 mM to 12 mM and NTPs at a concentration of 7.5 mM to 20 mM;
Mg²⁺ at a concentration of 9 mM to 15 mM and NTPs at a concentration of 11.3 mM to 25 mM;
Mg²⁺ at a concentration of 12 mM to 18 mM and NTPs at a concentration of 15 mM to 30 mM;
Mg²⁺ at a concentration of 15 mM to 21 mM and NTPs at a concentration of 18.8 mM to 35 mM;
Mg²⁺ at a concentration of 18 mM to 24 mM and NTPs at a concentration of 22.5 mM to 40 mM;
Mg²⁺ at a concentration of 21 mM to 27 mM and NTPs at a concentration of 26.25 mM to 45 mM;

In more preferred embodiments in that context, the molar ratio of Mg²⁺ to NTPs (the total NTP concentration as defined herein) in the IVT buffer is at about 0.7 and the respective Mg²⁺ and NTP concentrations are selected from:
Mg²⁺ at a concentration of 1 mM to 6 mM and NTPs at a concentration of 1.4 mM to 8.6 mM;
Mg²⁺ at a concentration of 3 mM to 9 mM and NTPs at a concentration of 4.3 mM to 12.9 mM;
Mg²⁺ at a concentration of 6 mM to 12 mM and NTPs at a concentration of 7.5 mM to 17.1 mM;
Mg²⁺ at a concentration of 9 mM to 15 mM and NTPs at a concentration of 8.6 mM to 21.4 mM;
Mg²⁺ at a concentration of 12 mM to 18 mM and NTPs at a concentration of 17.1 mM to 25.7 mM;
Mg²⁺ at a concentration of 15 mM to 21 mM and NTPs at a concentration of 21.4 mM to 30 mM;
Mg²⁺ at a concentration of 18 mM to 24 mM and NTPs at a concentration of 25.7 mM to 34.3 mM;
Mg²⁺ at a concentration of 21 mM to 27 mM and NTPs at a concentration of 30 mM to 38.6 mM;

### Cap Analogue/Cap Structure

In preferred embodiments, IVT buffer comprises a cap analogue, preferably a cap1 analogue.

The term “cap analogue” as used herein will be recognized and understood by the person of ordinary skill in the art, and is e.g. intended to refer to a non-polymerizable di-nucleotide or tri-nucleotide that has cap functionality in that it facilitates translation or localization, and/or prevents degradation of an RNA molecule when incorporated at the 5′-end of the nucleic acid molecule. Non-polymerizable means that the cap analogue will be incorporated only at the 5′-terminus because it does not have a 5′ triphosphate and therefore cannot be extended in the 3′-direction by a template-dependent polymerase, particularly, by template-dependent RNA polymerase.

In embodiments, the IVT buffer comprises a tri-nucleotide cap analogue as disclosed in WO2017053297, WO2017066793, WO2017066781, WO2017066791, WO2017066789, WO2017066782, WO2018075827, WO2017066797, and WO2023007019.

Preferably, cap structures derivable from the structure disclosed in claim 1-5 of WO2017053297 may be suitably contained in the IVT buffer.

Accordingly, in preferred embodiments, the cap1 analogues m7G(5′)ppp(5′)(2′OMeA)pG or m7G(5′)ppp(5′)(2′OMeG)pG are contained in the NTP mixture of the IVT buffer. A particularly preferred cap1 analogue in that context is m7G(5′)ppp(5′)(2′OMeA)pG.

In other preferred embodiments in that context, the cap1 analogue is 3′OMe-m7G(5′)ppp(5′)(2′OMeA)pG.

Alternatively, a cap1 analogue as described in WO2023007019 may be used.

Suitably, the IVT buffer comprises a cap analogue, preferably a cap1 analogue as defined herein, in a concentration of 1 mM to 10 mM, preferably 5 mM to 8 mM. In experiments provided herein, 5 mM to 8 mM of a Cap1 analog have been used.

### Antioxidants Such as Dithiothreitol (DTT)

Typically, IVT buffers comprise antioxidants such as Dithiothreitol (DTT) or TCEP to prevent an oxidation of the RNA polymerase. However, as described herein, large amounts of DTT may cause an unwanted agglomeration of the DNA beads and/or may impact the quality or integrity of DNA beads. For example, when beads comprise iron, e.g., as in the case of magnetic beads, DTT can potentially reduce said metal that can impact bead quality and hence efficiency and/or quality of the RNA synthesis.

In preferred embodiments, the IVT buffer comprises an antioxidant (e.g. DTT) at a concentration of less than 10 mM, preferably less than 5 mM, more preferably less than 2.5 mM, most preferably less than or about 1 mM. In preferred embodiments, the IVT buffer comprises an antioxidant (e.g. DTT) at a concentration ranging from 0.5 mM to 10 mM, preferably ranging from 0.5 mM to 5 mM, more preferably ranging from 0.5 mM to 2.5 mM, most preferably ranging from 0.5 mM to 1.5 mM. In a specific embodiment, the IVT buffer comprises about 1 mM an antioxidant (e.g. DTT).

Surprisingly, the performance of RNA in vitro transcription (e.g., RNA yield) using DNA beads is not impaired by a reduced antioxidant concentration in the IVT buffer, e.g., when DTT is present at about 1 mM.

### RNA Polymerase

IVT buffers comprise enzymes that facilitate the transcription from a DNA template into an RNA. Typically, DNA dependent RNA polymerases are used. Suitable RNA polymerases in the context of the invention may be selected from bacteriophage derived RNA polymerases, for example T7, T3, SP6, or Syn5 RNA polymerases. These RNA polymerases may be engineered to e.g. improve the quality of the synthetized RNA (e.g. reduced dsRNA content, reduced short abortive by-products, improved capping efficiency).

The suitable amount of RNA polymerases in the RNA in vitro transcription composition of the invention may have to be adapted based on the fact that the DNA templates are immobilized on beads. As proteins in the RNA in vitro transcription composition may promote an agglomeration of DNA beads, the amount of RNA polymerase has to be adapted to allow sufficient RNA synthesis.

In preferred embodiments, the IVT buffer comprises at least 5 units/ml RNA polymerase, preferably at least 10 units/ml RNA polymerase. Suitably, the IVT buffer comprises RNA polymerase in a range of 5 units/ml to 20 units/ml, preferably 10 units/ml to 20 units/ml, most preferably 10 units/ml to 15 units/ml. In specific embodiments, the IVT buffer comprises about 12.5 units/ml RNA polymerase. In experiments provided herein, 10 units/ml to 15 units/ml of T7 RNA polymerase have been used.

In preferred embodiments, the RNA polymerase is selected or derived from a T7, T3, SP6, or Syn5 RNA polymerase. In particularly preferred embodiments, the RNA polymerase is selected or derived from a T7 RNA polymerase.

In embodiments, the IVT buffer may comprise additional components to e.g., prevent or reduce RNA degradation (e.g., RNAse), to reduce or prevent the formation of pyrophosphate (e.g., Pyrophosphatase), to e.g. maintain a preferred pH (e.g. buffering agents), to resolve secondary structures in the DNA and/or the RNA (e.g. betaine).

In embodiments, the IVT buffer additionally comprises an RNAse inhibitor, a pyrophosphatase (PPase), and/or a buffering agent.

Preferably, the IVT buffer comprises 0.1-0.3 units/μl RNAse inhibitor.

Preferably, the IVT buffer comprises 0.003-0.01 units/μl PPase.

Preferably, the IVT buffer comprises a buffering agent selected from a phosphate buffer agent, a Tris buffer agent, a borate buffer agent, a succinate buffer agent, a histidine buffer agent, a HEPES buffer agent, a citrate buffer agent. In preferred embodiments, the IVT buffer comprises Tris (e.g. Tris-HCl) as a buffering agent.

Preferably, the IVT buffer comprises Tris as a buffering agent in a concentration of about 30 mM to 150 mM, preferably 50 mM to 100 mM, in particular in a concentration of about 80 mM (e.g. 80 mM Tris-HCl).

Suitably, the pH of the IVT buffer is at about pH 7.5 to pH 8.5, in particular at about pH 8.0.

In the context of a pharmaceutical RNA production, it is preferred to reduce the number of components in the RNA in vitro transcription composition. Accordingly, in preferred embodiments, the RNA in vitro transcription composition does not comprise PEG. In preferred embodiments, the RNA in vitro transcription composition does not comprise BSA. In preferred embodiments, the RNA in vitro transcription composition does not comprise a Tween-20. In preferred embodiments, the RNA in vitro transcription composition does not comprise Triton. In preferred embodiments, the RNA in vitro transcription composition does not comprise NaCl.

According to preferred embodiments, the RNA in vitro transcription composition that comprises DNA templates immobilized on beads are contained in an RNA in vitro transcription (IVT) buffer, wherein the IVT buffer is characterized by at least one, at least two, or a combination of the following features F1 to F6
F1: less than 1 mM spermidine, preferably free of spermidine;
F2: Mg²⁺ at a concentration between 1 mM to 25 mM, preferably less than 20 mM Mg²⁺;
F3: the added total concentration of spermidine and Mg²⁺ in the IVT buffer is less than 25 mM;
F4: the molar ratio of Mg²⁺ to NTPs is ranging from 1.2 to 0.5, preferably ranging from 0.8 to 0.6;
F5: NTPs at a total concentration of at least 3 mM, preferably ranging from 3 mM to 35 mM;
F6: less than 10 mM DTT, preferably less than or about 1 mM DTT;
   wherein the IVT buffer is preferably configured to reduce or prevent agglomeration of the DNA beads e.g. during the course of RNA in vitro transcription.

In preferred embodiments in that context, the IVT buffer of the RNA in vitro transcription composition is characterized by the following features, sorted by increasing preference:
F1 and F2
F1 and F2 and F3
F1 and F2 and F4
F3 and F4
F1 and F2 and F3 and F4
F3 and F5 or F2 and F5
F1 and F2 and F3 and F4 and F5
F1 and F2 and F3 and F4 and F5 and F6

### DNA Beads

The RNA in vitro transcription composition of the invention comprises DNA templates that are immobilized on beads (DNA beads) or particles, wherein the DNA beads serve as a template for RNA in vitro transcription as defined herein. DNA templates immobilized on beads are particularly suitable in the context of an automated pharmaceutical RNA production as DNA beads can be re-used for several IVT cycles which increases the RNA yield of the RNA production process. Furthermore, DNA beads may be removed easily (e.g., by filtration or magnetic forces) and, therefore, the DNA used for RNA synthesis does not contaminate the final RNA product. As described above, the IVT buffer of the RNA in vitro transcription composition is configured to reduce or prevent agglomeration of the DNA beads. In addition, the DNA beads used herein can also have certain preferred characteristics to optimize the RNA synthesis. These preferred characteristics are specified below.

The term “bead” or “particle” in the context of the invention has to be understood as solid supports that are suitable for the use in RNA in vitro transcription. Accordingly, the term “bead” as such is not limiting in scale, shape, or composition. However, “beads” in the context of the invention have to be understood as free floating solid supports (e.g. free floating upon mixing, shaking etc.).

In the context of the invention, the DNA serves as a template for RNA in vitro transcription (IVT). Accordingly, during IVT, the DNA sequence is transcribed into an RNA sequence. Therefore, the DNA template may comprise certain sequence elements, depending on which type of RNA is to be produced. Preferably, the DNA template codes for (that is, serves as a template for) any type of therapeutic RNA, preferably an mRNA, a replicon RNA, or a circular RNA.

The DNA template typically comprises a spacer sequence at the 5′ end (a DNA sequence element at the 5′ terminus that extends the separation from the bead), a promoter sequence for an RNA polymerase (e.g. T7 promoter, SP6 promoter), and can comprise further elements selected from a Kozak sequence, an IRES, UTR sequences (3′ UTR and/or 5′ UTR), a coding sequence (cds), a Poly(A/T)sequence (e.g. comprising about 100 A/T nucleotides), or a histone-stem loop.

In preferred embodiments, the DNA template can comprise the following elements in the following order:
spacer sequence at the 5′ end;
RNA polymerase promoter, e.g., T7 promoter;
5′ UTR element;
Translation initiation sequence (e.g., Kozak and/or IRES);
coding sequence (cds) encoding at least one peptide or protein;
3′ UTR element;
Poly(A/T)sequence

The amount of DNA templates (or DNA beads) in the RNA in vitro transcription composition of the invention may have to be adapted to allow for an efficient RNA synthesis without causing bead agglomeration.

In preferred embodiments, the RNA in vitro transcription composition comprises 10 μg/ml to 100 μg/ml immobilized DNA templates, preferably 20 μg/ml to 80 μg/ml immobilized DNA templates, more preferably 50 μg/ml to 70 μg/ml immobilized DNA templates. In specific embodiments, the RNA in vitro transcription composition comprises about 60 μg/ml immobilized DNA templates.

In embodiments, the RNA in vitro transcription composition is essentially free of non-immobilized DNA templates which means that the RNA in vitro transcription composition contains about 90% immobilized DNA templates and about 10% non-immobilized DNA templates, preferably the RNA in vitro transcription composition contains about 95% immobilized DNA templates and about 5% non-immobilized DNA templates, more preferably the RNA in vitro transcription composition contains about 99% or more than 99% immobilized DNA templates, and about 1% or less than 1% non-immobilized DNA templates.

In preferred embodiments, the RNA in vitro transcription composition is essentially free of non-immobilized DNA or DNA templates.

It is particularly preferred and advantageous that the RNA in vitro transcription composition is essentially free of non-immobilized DNA templates to avoid a contamination of the produced RNA. Accordingly, also after having performed the RNA in vitro transcription, it is preferred that the DNA template is immobilized on the bead.

Accordingly, in preferred embodiments, upon performing an RNA in vitro transcription reaction for at least 1 h at about 37° C., the RNA in vitro transcription composition is essentially free of non-immobilized DNA templates.

“Non-immobilized DNA templates” encompass all DNA species that are suitable for RNA in vitro transcription and that are not immobilized on a bead as defined herein.

In preferred embodiments, the RNA in vitro transcription composition is essentially free of other DNA species including bacterial genomic DNA, bacterial plasmid DNA, viral genomic DNA, or eucaryotic DNA.

In preferred embodiments, the RNA in vitro transcription composition comprises 1 mg/ml to 100 mg/ml DNA beads, preferably 10 mg/ml to 50 mg/ml DNA beads, more preferably 20 mg/ml to 40 mg/ml DNA beads. In specific embodiments, the RNA in vitro transcription composition comprises about 35 mg/ml DNA beads.

The DNA beads of the RNA in vitro transcription composition may have certain characteristics that may be advantageous in the context of the invention. For example, the DNA density on the beads, the bead diameter, or the specific bead material may be adjusted to optimize for an efficient RNA synthesis and/or to reduce or prevent an agglomeration of the DNA beads.

In preferred embodiments, the DNA beads comprise about 1 mg DNA per 0.3 g to 0.8 g beads. In specific embodiments, the DNA beads comprise about 1 mg DNA per 0.6 g beads.

A certain density of DNA templates immobilized on the surface of the beads may be advantageous in the context of the present invention. Accordingly, a certain amount of DNA per mm² bead surface may be suitable.

Accordingly, in preferred embodiments, the DNA bead surface comprise about 0.5 ng DNA/mm² to about 10 ng DNA/mm², preferably about 1 ng DNA/mm² to about 5 ng DNA/mm², most preferably about 1.4 ng to 3.1 ng DNA/mm².

The density of DNA on the bead surface is a factor that can influence the yield of the IVT and the DNA beads agglomeration. Increasing the density of DNA on the bead surface to more than e.g. 10 ng DNA/mm² may have a negative impact on IVT performance and/or DNA beads agglomeration.

The beads on which the DNA template is immobilized is preferably spherical in shape and essentially uniform in diameter. The bead diameter may range from 500 nm to 50 μm. However, in the context of using the RNA in vitro transcription composition in a pharmaceutical RNA production process, it may be advantageous that the bead diameter is in a certain preferred range to reduce the risk of contaminating the final RNA product with beads (e.g. in case beads are selected that are below a certain diameter) or to reduce the risk of bead agglomeration (e.g. in case beads are selected that are above a certain diameter).

In preferred embodiments, the beads have a (uniform) diameter in a range of 1 μm to 10 μm, preferably in a range of 1 μm to 5 μm, more preferably in a range of 4 μm to 5 μm, even more preferably in a range of 4.3 μm to 4.7 μm. In preferred embodiments, the beads have a (uniform) diameter of about 4.5 μm.

In other embodiments, the beads have a (uniform) diameter of about 2.8 μm. In other embodiments, the beads have a (uniform) diameter of about 1 μm.

To allow the removal of the beads via commercially available filter systems (e.g. using 0.2 μm filtration) e.g. after having carried out an RNA in vitro transcription reaction, it may be preferred to use beads with a size larger than 1 μm, preferably larger than 2 μm.

Suitably, the size distribution of the beads is uniform, that means that the size distribution has a CV value of smaller than 5%. Size distribution may be measured using a multisizer device (Beckman Coulter).

The beads on which the DNA template is immobilized can comprise materials selected or derived from iron oxide, silica, cellulose, sepharose, sephadex, polystyrene, agarose, (poly)methacrylate, poly(methyl)methacrylate, or any derivative or combination thereof.

Preferably, the beads on which the DNA template is immobilized are non-porous.

Preferably, the beads on which the DNA template is immobilized have a density ranging from 1 g/cm³ to 2 g/cm³, 1.3 g/cm³ to 1.5 g/cm³, preferably a density of about 1.4 g/cm³.

In preferred embodiments, the beads on which the DNA template is immobilized comprise polystyrene or a derivative of polystyrene.

In various embodiments, the RNA in vitro transcription composition is configured and suitable for magnetic mixing and/or magnetic capturing. Accordingly, the beads on which the DNA template is immobilized comprise a magnetizable material, preferably a (super) paramagnetic material. Suitably, the beads comprise magnetic iron oxide, preferably about 10% to 50% magnetic iron oxide (e.g., gamma-Fe2O3 and/or magnetite (Fe3O4)).

In preferred embodiments, the beads on which the DNA template is immobilized are magnetic beads, preferably (super) paramagnetic beads. Accordingly, the DNA beads of the RNA in vitro transcription composition are magnetic DNA beads, preferably (super) paramagnetic DNA beads. Advantageously, magnetic DNA beads allow for a mixing of the DNA beads during the course of the IVT and/or a capturing of the DNA beads after the IVT cycle which is important for an automated RNA manufacturing process. The mixing and/or capturing is preferably induced by magnetic force, e.g., by a magnet.

In preferred embodiments, the beads on which the DNA template is immobilized are functionalized with streptavidin (to bind to biotin). In embodiments in that context, the beads comprise about 25 to 400 μmol biotin binding capacity per mg beads, preferably about 50 to 300 binding capacity per mg beads

Preferred examples of beads that are suitable in the context of the invention are Dynabeads® such as Dynabeads® M-270, Dynabeads® M-280, Dynabeads® M-450, or Dynabeads® MyOne.

In specific embodiments, the beads on which the DNA template is immobilized are Dynabeads® M-450, e.g. Dynabeads® M-450 E beads.

Preferred in the context of the invention is that the linear DNA template is immobilized via one DNA terminus to the bead, and that the other terminus of the DNA template is free (which means that this free terminus is not immobilized on a bead). Suitably, the DNA template is immobilized on the DNA bead to allow a run-off RNA in vitro transcription, wherein the free end of the DNA template defines the termination of the RNA transcription reaction (e.g., as illustrated in FIG. 1B of WO2019122371).

In preferred embodiments, DNA beads comprise a biotin-streptavidin immobilized DNA template, preferably a linear biotin-streptavidin immobilized DNA template. As described above, the DNA template is preferably immobilized to allow a run-off RNA in vitro transcription (e.g., as illustrated in FIG. 1B of WO2019122371).

In preferred embodiments, the DNA beads have been produced by site specific immobilization of a biotinylated PCR amplified DNA template on a streptavidin functionalized bead, or by site specific immobilization of a biotinylated linearized plasmid DNA template on a streptavidin functionalized bead.

For immobilizing a linearized plasmid DNA template, a method disclosed in WO2019122371 may be used, preferably a method as defined in claims 6 to 29 of WO2019122371. The biotinylated linearized plasmid DNA template may suitably be immobilized on a streptavidin functionalized bead.

For immobilizing a PCR amplified DNA template, a biotinylated PCR primer may be used in the PCR-based DNA template production. The biotinylated PCR amplified DNA template may suitably be immobilized on a streptavidin functionalized bead.

Preferably, the DNA beads of the invention comprise biotin-streptavidin immobilized DNA templates. Preferably, these biotin-streptavidin immobilized DNA templates have been produced by site specific immobilization of a biotinylated PCR amplified DNA template or a biotinylated linearized plasmid DNA template on a streptavidin functionalized bead to allow a run-off RNA in vitro transcription.

The DNA used for immobilization is preferably a purified DNA, for example an RP-HPLC purified DNA. Alternatively, the DNA may be purified by normal phase chromatography, mixed-mode chromatography, anion exchange chromatography, or size exclusion chromatography.

Suitably, a DNA bead of the invention may comprise the following structural elements:

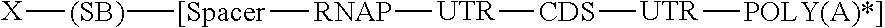

wherein X is a bead, preferably a magnetic bead; SB is a streptavidin-biotin linkage; Elements between the rectangular brackets [ ] represent DNA elements; Spacer is a DNA sequence element at the 5′ terminus that extends the separation from the bead; RNAP is an RNA polymerase promotor sequence; UTR is an untranslated region; CDS is a coding sequence; POLY(A) is a poly(A) sequence; Asterisks indicates the free end of the DNA.

According to preferred embodiments, the DNA beads are characterized by at least one, at least two, or a combination of the following features B1 to B5
B1: comprise a magnetizable material, preferably a (super) paramagnetic material;
B2: diameter in a range of 1 μm to 10 μm, preferably in a range of 4 μm to 5 μm, e.g. 4.5 μg;
B3: comprise polystyrene or a derivative of polystyrene;
B4: the bead surface loading comprises about 1 ng DNA/mm² to about 5 ng DNA/mm²
B5: beads comprise a biotin-streptavidin immobilized DNA template;

In preferred embodiments in that context, the IVT buffer of the RNA in vitro transcription composition is characterized by the following features, sorted by increasing preference:
B1 and B2;
B1 and B2 and B3;
B1 and B2 and B3 and B4;
B1 and B2 and B3 and B4 and B5;

In embodiments, the RNA in vitro transcription composition is configured to produce more than one RNA species, e.g. 2, 3, 4, 5, 6, 7, 8, 9, or even more RNA species. In such embodiments, the RNA in vitro transcription composition comprises more than one, e.g. 2, 3, 4, 5, 6, 7, 8, 9, or even more different DNA templates immobilized on beads. These 2, 3, 4, 5, 6, 7, 8, 9, or even more different DNA templates comprise a cds that codes for a different peptide or protein (e.g. different tumor antigens, different viral antigens etc.).

In preferred embodiments, the composition is configured to reduce or prevent attachment of RNA to the DNA beads at RNA concentrations of more than 100 mg/L, preferably at RNA concentrations of more than 500 mg/L, more preferably at RNA concentrations of more than 1 g/L.

The RNA in vitro transcription composition of the invention inter alia solves the as yet undescribed problem of DNA bead agglomeration during the course of the RNA in vitro transcription. That observed problem is solved by adjusting the components of the RNA in vitro transcription as described herein. Notably, the inventors observed that the problem of DNA beads agglomeration that is occurring during the course of RNA in vitro transcription can be dependent on the RNA concentration (that is, the RNA that is constantly produced during the IVT), the duration of the RNA in vitro transcription, or the fact that the DNA beads of the RNA in vitro transcription composition is used for several IVT cycles.

In preferred embodiments, the RNA in vitro transcription composition is configured to reduce or prevent DNA bead agglomeration at RNA concentrations of more than 0.1 g/L, preferably at RNA concentrations of more than 1 g/L, more preferably at RNA concentrations of more than 5 g/L. In that context it has to be understood that the specified RNA concentrations are the product of the RNA synthesis that occurs during the RNA in vitro transcription. Agglomeration can be determined and quantified by microscopy.

In preferred embodiments, the RNA in vitro transcription composition is configured for a repetitive use of the DNA beads, in particular for a use of more than one IVT reaction or cycle, e.g., 2, 3, 4, 5, 6, or more IVT cycles. Preferably one IVT cycle is performed for at least 1 h at about 37° C. After each IVT cycle, the IVT buffer is typically replenished while the DNA beads remain in the composition.

In preferred embodiments, upon incubation of the DNA beads for more than one IVT reaction or cycle, e.g., 2, 3, 4, 5, 6, or more IVT cycles, each of the more than one IVT reaction or cycle, e.g., 2, 3, 4, 5, 6, or more IVT cycles yields essentially the same amount of RNA (given that the conditions of each IVT cycle is essentially the same).

In preferred embodiments, the RNA in vitro transcription composition is configured for an incubation period of more than 1 h, more than 2 h, more than 3 h, more than 4 h, preferably at temperatures of at about 37° C.

In preferred embodiments, upon incubation of the RNA in vitro transcription composition for at least 1 h, less than 10% of the DNA beads contained in the composition are agglomerated, preferably less than 5% of the DNA beads contained in the composition are agglomerated, more preferably less than 1% of the DNA beads contained in the composition are agglomerated. In preferred embodiments, the incubation temperature is at about 37° C. Agglomeration can be determined and quantified by microscopy.

In preferred embodiments, the RNA in vitro transcription composition is essentially free of pyrogens, bacteria or fragments thereof, viruses or fragments thereof, and/or phages or fragments thereof. Preferably, the RNA in vitro transcription composition is compliant for use in a pharmaceutical production, e.g., in a GMP (guidelines for good manufacturing) production.

In preferred embodiments, the RNA in vitro transcription composition is for producing any type of RNA as defined herein, preferably any type of therapeutic RNA as defined herein. In preferred embodiments RNA in vitro transcription composition is for producing (therapeutic) mRNA.

### Use of the RNA In Vitro Transcription Composition:

In a second aspect, the present invention provides the use of the RNA in vitro transcription composition as defined in the context of the first aspect in a manufacturing process of RNA.

It has to be noted that features and embodiments that are described in the context of the RNA in vitro transcription composition of the first aspect may also be applicable to the use of the second aspect. Likewise, features and embodiments that are described in the context of the use of the second aspect may also be applicable to the RNA in vitro transcription composition of the first aspect.

In preferred embodiments in that context, the manufacturing process is an automated RNA manufacturing process. In preferred embodiments in that context, the manufacturing process is a GMP (guidelines for good manufacturing practice) compliant RNA manufacturing process. In preferred embodiments in that context, the manufacturing process is a process that produces more than 1 mg of RNA, preferably more than 1 g of RNA.

In preferred embodiments in that context, the RNA in vitro transcription composition as defined in the context of the first aspect may be used in a manufacturing process of any type of RNA as defined herein. Preferably, the RNA in vitro transcription composition as defined in the context of the first aspect may be used in a manufacturing process of any type of therapeutic RNA as defined herein. Most preferably, the RNA in vitro transcription composition as defined in the context of the first aspect may be used in a manufacturing process of any type of therapeutic coding RNA as defined herein, preferably mRNA, for example an mRNA for an infectious disease vaccine or a tumour vaccine.

### Method for Producing RNA:

In a third aspect, the present invention provides a method for producing RNA using an RNA in vitro transcription composition comprising DNA templates immobilized on beads.

It has to be noted that features and embodiments that are described in the context of the RNA in vitro transcription composition of the first aspect may also be applicable to the RNA production method of the third aspect. Likewise, features and embodiments that are described in the context of the RNA production method of the third aspect may also be applicable to the RNA in vitro transcription composition of the first aspect.

In various embodiments, the RNA production method comprises the step of incubating an RNA in vitro transcription composition under conditions to allow an RNA transcription, wherein the RNA in vitro transcription composition comprises DNA templates immobilized on beads that are contained in an RNA in vitro transcription (IVT) buffer. Preferably, the IVT buffer is configured to reduce or prevent agglomeration of the DNA beads during the incubation (that is, during the course of RNA transcription).

The method is suitable for producing any type of RNA.

In embodiments, the RNA is selected from a single stranded RNA or double stranded RNA, and/or a coding RNA or a non-coding RNA, and/or a linear RNA or a circular RNA. Accordingly, the RNA may be a double-stranded non-coding RNA in circular form, or the RNA may be a single stranded non-coding RNA in linear form, or the RNA may be a double stranded coding RNA in linear form, etc. Suitably, the RNA is a single stranded coding RNA in linear or circular form.

In embodiments, the RNA is selected from viral RNA, retroviral RNA, replicon RNA, small interfering RNA (siRNA), antisense RNA, saRNA (small activating RNA), CRISPR RNA (small guide RNA, sgRNA), ribozymes, aptamers, riboswitches, immunostimulating RNA, transfer RNA (tRNA), ribosomal RNA (rRNA), small nuclear RNA (snRNA), small nucleolar RNA (snoRNA), microRNA (miRNA), Piwi-interacting RNA (piRNA), self-replicating RNA, circular RNA, or messenger RNA (mRNA).

In preferred embodiments, the RNA is a coding RNA. In preferred embodiments, the RNA comprises at least one coding sequence.

A coding RNA can be any type of RNA characterized in that said RNA comprises at least one coding sequence (cds) that is translated into at least one amino-acid sequence (upon administration to e.g., a cell).

In preferred embodiments, the RNA is selected from an mRNA, a (coding) circular RNA, a (coding) self-replicating RNA, a (coding) viral RNA, or a (coding) replicon RNA.

In embodiments, the RNA has a length ranging from about 500 nucleotides to about 10000 nucleotides, ranging from about 1000 nucleotides to about 10000 nucleotides, ranging from about 1500 nucleotides to about 5000 nucleotides.

The problem of DNA bead agglomeration may be more pronounced for producing RNA that has a certain length. In preferred embodiments, the method for producing RNA is a method for producing RNA that has a length of at least 1000 nucleotides, preferably at least 1500 nucleotides, more preferably at least 2000 nucleotides. In particular, the RNA has a length ranging from 1500 nucleotides to about 5000 nucleotides.

In particularly preferred embodiments, the RNA is an mRNA. Accordingly, the method is a method for producing coding RNA, preferably mRNA. In preferred embodiments, the RNA is a therapeutic RNA.

The term “therapeutic RNA” relates to an RNA providing a therapeutic modality. The term “therapeutic” in that context has to be understood as “providing a therapeutic function” or as “being suitable for therapy or administration”. Moreover, a “therapeutic RNA” is typically produced using methods and compositions suitable for pharmaceutical production. However, “therapeutic” in that context should not at all to be understood as being limited to a certain therapeutic modality. Typically, the term “therapeutic RNA” does not include natural RNA extracts or RNA preparations (e.g., obtained from bacteria, or obtained from plants) that are not suitable for administration to a subject (e.g., animal, human).

In preferred embodiments, the RNA is an artificial RNA.

The term “artificial RNA” as used herein is intended to refer to an RNA that does not occur naturally. In other words, an artificial RNA may be understood as a non-natural RNA molecule. Such RNA molecules may be non-natural due to their individual sequence (e.g., G/C content modified coding sequence, UTRs) and/or due to other modifications, e.g., structural modifications of nucleotides. Typically, artificial RNA may be designed and/or generated by genetic engineering to correspond to a desired artificial sequence of nucleotides. In this context, an artificial RNA is a sequence that may not occur naturally, i.e., a sequence that differs from the wild type or reference sequence/the naturally occurring sequence by at least one nucleotide (via e.g., codon modification as further specified below). The term “artificial RNA” is not restricted to mean “one single molecule” but is understood to comprise an ensemble or plurality of essentially identical RNA molecules.

It has been shown that the problem of DNA bead agglomeration may be more pronounced for producing RNA that has a certain G/C content or that comprises a coding sequence that has an increased or maximized GC content. In this context, the disclosure of WO2002098443 is included in its full scope in the present invention. G/C optimized coding sequences are indicated by the abbreviations “gc”. In embodiments, the RNA sequence or the coding sequence has a G/C content of at least about 50%, 55%, or 60%. In particular embodiments, the RNA or the coding sequence has a G/C content of at least about 50%, 51%, 52%, 53%, 54%, 55%, 56%, 57%, 58%, 59%, 60%, 61%, 62%, 63%, 64%, 65%, 66%, 67%, 68%, 69%, or 70%.

Accordingly, in preferred embodiments, the method for producing RNA is a method for producing RNA that has an RNA sequence or the coding sequence with a G/C content of at least about 55%.

In preferred embodiments, the produced RNA comprises at least one 3′UTR and/or at least one 5′UTR sequence. Suitable UTR sequences and UTR combinations may be selected from published PCT patent application WO2019077001, preferably selected from UTR sequences and UTR combinations as disclosed in claims 1 to 10 of WO2019077001. Preferably, the 5′ UTR is selected or derived from HSD17B4, and the 3′ UTR is selected or derived from PSMB3. In preferred embodiments, the produced RNA comprises at least one histone stem loop sequence (hSL). Suitable hSL sequences that may be used within the present invention may be derived from formulae (I) or (II) of WO2012019780. In preferred embodiments, the produced RNA comprises at least one poly(A) sequence or at least two poly(A) sequences (see e.g., WO2016091391). In preferred embodiments, the poly(A) sequences represents the 3′ terminus of the RNA (see e.g., WO2022162027). Suitably, the Poly(A) sequence has a length of about 100 A. In some embodiments, the Poly(A) sequence has a length of about 64 A.

In preferred embodiments, the method for producing RNA is a method for producing therapeutic coding RNA (e.g., therapeutic mRNA).

Suitably, therapeutic coding RNA, preferably the mRNA comprises at least one coding sequence that encodes at least one therapeutic peptide or protein.

In embodiments in that context, the at least one therapeutic peptide or protein is selected or derived from an antibody, an intrabody, a receptor, a receptor agonist, a receptor antagonist, a binding protein, a CRISPR-associated endonuclease, a chaperone, a transporter protein, an ion channel, a membrane protein, a secreted protein, a transcription factor, an enzyme, a peptide or protein hormone, a growth factor, a structural protein, a cytoplasmic protein, a cytoskeletal protein, a viral antigen or epitope, a bacterial antigen or epitope, a protozoan antigen or epitope, an allergen, a tumor antigen or epitope, or fragments, variants, or combinations of any of these.

In embodiments in that context, the at least one therapeutic peptide or protein is selected or derived from an antigen or epitope of a pathogen (e.g. a viral antigen or epitope, a bacterial antigen or epitope, a protozoan antigen or epitope) or from an antigen or epitope of a tumor.

In preferred embodiments of the third aspect, the method for producing RNA comprises the steps of
(A) Providing an RNA in vitro transcription composition comprising DNA beads in an IVT buffer contained in a reaction vessel; and
(B) Performing a first or initial IVT cycle by mixing the DNA beads with the IVT buffer to produce an RNA batch.

According to preferred embodiments, the RNA in vitro transcription composition of the method is characterized by any of the features of the first aspect.

Suitably, the RNA in vitro transcription composition comprises DNA templates immobilized on beads contained in an RNA in vitro transcription (IVT) buffer, wherein the IVT buffer preferably comprises less than 2 mM spermidine; less than 25 mM Mg²⁺; and Mg²⁺ and NTP in a molar ratio of below 1.4.

Preferably, the RNA in vitro transcription composition of the method comprises DNA templates immobilized on beads (DNA beads) that are contained in an RNA in vitro transcription (IVT) buffer, wherein the IVT buffer is characterized by at least one, at least two, or a combination of the following features F1 to F6
F1: less than 1 mM spermidine, preferably free of spermidine;
F2: Mg²⁺ at a concentration between 1 mM to 25 mM, preferably less than 20 mM Mg²⁺;
F3: the added total concentration of spermidine and Mg²⁺ in the IVT buffer is less than 30 mM;
F4: the molar ratio of Mg²⁺ to NTPs is ranging from 1.2 to 0.5, preferably ranging from 0.8 to 0.6;
F5: NTPs at a total concentration of at least 3 mM, preferably ranging from 3 mM to 35 mM;
F6: less than 10 mM DTT, preferably less than or about 1 mM DTT;
   wherein the IVT buffer is preferably configured to reduce or prevent agglomeration of the DNA beads e.g. during the course of RNA in vitro transcription, at high RNA concentrations, or at repetitive IVT cycles.

In preferred embodiments in that context, the IVT buffer of the RNA in vitro transcription composition used in the method is characterized by the following features, sorted by increasing preference:
F1 and F2
F1 and F2 and F3
F1 and F2 and F4
F3 and F4
F1 and F2 and F3 and F4
F3 and F5 or F2 and F5
F1 and F2 and F3 and F4 and F5
F1 and F2 and F3 and F4 and F5 and F6

In preferred embodiments of the method, the RNA in vitro transcription composition comprises 10 μg/ml to 100 μg/ml immobilized DNA templates, preferably 20 μg/ml to 80 μg/ml immobilized DNA templates, more preferably 35 μg/ml to 65 μg/ml immobilized DNA templates. In specific embodiments, the RNA in vitro transcription composition comprises about 50 μg/ml immobilized DNA templates.

In preferred embodiments of the method, the RNA in vitro transcription composition is essentially free of non-immobilized DNA templates. In preferred embodiments of the method, the RNA in vitro transcription composition is essentially free of other DNA species including bacterial genomic DNA, viral genomic DNA, or eucaryotic DNA.

In preferred embodiments of the method, the RNA in vitro transcription composition comprises 1 mg/ml to 100 mg/ml DNA beads, preferably 10 mg/ml to 50 mg/ml DNA beads, more preferably 20 mg/ml to 40 mg/ml DNA beads. In specific embodiments, the RNA in vitro transcription composition comprises about 35 mg/ml DNA beads.

The method of the present aspect is optimized and adapted to allow an effective use of DNA templates immobilized on beads (DNA beads), to inter alia reduce or prevent an unwanted DNA beads agglomeration and/or an unwanted DNA beads escape.

Preferred DNA beads that are particularly suitable in the method have been described in the context of the first aspect. Notably, features relating to DNA beads provided in the context of the first aspect may likewise be applied to the DNA beads of the method of the third aspect.

Particularly preferred embodiments of DNA beads are summarized in the following.

According to preferred embodiments of the method, the DNA beads are characterized by at least one, at least two, or a combination of the following features B1 to B5
B1: comprise a magnetizable material, preferably a (super) paramagnetic material;
B2: diameter in a range of 1 μm to 10 μm, preferably in a range of 4 μm to 5 μm, e.g. 4.5 μg;
B3: comprise polystyrene or a derivative of polystyrene;
B4: the bead surface comprises about 1 ng DNA/mm² to about 5 ng DNA/mm²
B5: beads comprise a biotin-streptavidin immobilized DNA template;

In preferred embodiments in that context, the DNA beads used in the method are characterized by the following features, sorted by increasing preference:
B1 and B2;
B1 and B2 and B3;
B1 and B2 and B3 and B4;
B1 and B2 and B3 and B4 and B5;

In preferred embodiments of the method, the beads on which the DNA template is immobilized are magnetic beads, preferably (super) paramagnetic beads. Advantageously, magnetic DNA beads allow for a mixing of the DNA beads during the course of the IVT and/or a capturing of the DNA beads after the IVT cycle which is important for an automated RNA manufacturing process. The mixing and/or capturing is preferably induced by magnetic force, e.g., by a magnet as further specified herein.

Preferred examples of beads that are suitable in the context of the invention are Dynabeads® such as Dynabeads® M-270, Dynabeads® M-280, Dynabeads® M-450, or Dynabeads® MyOne.

In specific embodiments of the method, the beads on which the DNA template is immobilized are Dynabeads® M-450, e.g. Dynabeads® M-450 E beads.

In preferred embodiments of the method, DNA beads comprise a biotin-streptavidin immobilized DNA template, preferably a linear biotin-streptavidin immobilized DNA template. As described above, the DNA template is preferably immobilized to allow a run-off RNA in vitro transcription (e.g., as illustrated in FIG. 1B of WO2019122371).

In preferred embodiments of the method, the DNA beads have been produced by site specific immobilization of a biotinylated PCR amplified DNA template on a streptavidin functionalized bead, or by site specific immobilization of a biotinylated linearized plasmid DNA template on a streptavidin functionalized bead.

Accordingly, the DNA beads of the method comprise biotin-streptavidin immobilized DNA templates.

Suitably, a DNA bead of the method may comprise the following structural elements:

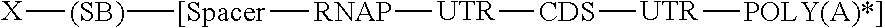

wherein X is a bead, preferably a magnetic bead; SB is a streptavidin-biotin linkage; Elements between the rectangular brackets [ ] represent DNA elements; Spacer is a DNA sequence element at the 5′ terminus that extends the separation from the bead; RNAP is an RNA polymerase promotor sequence; UTR is an untranslated region; CDS is a coding sequence; POLY(A) is a poly(A) sequence; Asterisks indicates the free end of the DNA.

In the context of the invention, a reaction vessel can be a microchannel, a tube, a vial, a container, or a bioreactor. Suitably, the reaction vessel is a reaction vessel that can be used for pharmaceutical production (e.g. GMP compliant). The reaction vessel can be composed of materials selected from glass, metal, ceramics, or a polymer (e.g. plastic material). Preferred materials are metal and ceramics. The reaction vessel can have a volume (e.g. the volume in which the RNA in vitro transcription composition is contained) of 1 μl to 10001, preferably 1 ml to 101, more preferably 10 ml to 11, even more preferably 10 ml to 500 ml.

In preferred embodiments, the reaction vessel is a bioreactor that is composed of metal or ceramics and has a volume of at least 1 ml, preferably 10 ml to 500 ml. In a specific embodiment, the reaction vessel is a bioreactor that is composed of ceramics and is configured to hold at least 50 ml.

In the context of the invention, an RNA batch has to be understood as the produced RNA that is synthesized during the process of IVT. The RNA batch may comprise more than 1 mg of RNA, for example 1 mg to 1000 g of RNA, preferably 1 mg to 100 g of RNA, more preferably 10 mg to 10 g of RNA. The RNA batch is preferably free of DNA-beads or fragments thereof.

In preferred embodiments, the method for producing RNA is configured to achieve high RNA concentrations in the IVT (or the IVT cycle). Suitably, concentrations of more than 100 mg/L, preferably at RNA concentrations of more than 1 g/L, more preferably at RNA concentrations of more than 5 g/L are achieved. Preferably, during the course of the method, DNA-beads agglomeration is reduced or prevented at RNA concentrations of more than 100 mg/L, preferably at RNA concentrations of more than 1 g/L, more preferably at RNA concentrations of more than 5 g/L.

In the context of the invention, an IVT cycle has to be understood as one RNA in vitro reaction round that is needed to in vitro transcribe RNA at the desired yield. Typically, an IVT cycle is performed over a period of at least 1 h (e.g. at least 1 h at about 37° C.). Typically, after performing the IVT cycle, the free NTPs are essentially consumed and integrated into the produced RNA molecules. In the context of the invention, the method for producing RNA may comprise more than one IVT cycle, preferably by using the same DNA-beads as templates and by feeding fresh IVT buffer into the reaction vessel as further outlined below.

In preferred embodiments, each IVT cycle of the method is performed at controlled temperature conditions

Suitably, IVT cycles of the method are carried out at 20-40° C., such as 30° C. to 40° C., preferably 36° C. to 38° C.

In certain embodiments, IVT cycles are is carried at about 37° C.

In preferred embodiments, IVT cycles are carried out for at least 1 h, 2 h, 3 h, 4 h, e.g. for 90 min to 180 min. In certain embodiments, each IVT cycle is carried out for about 2 h.

After performing Step (A) and (B) the RNA batch may be harvested.

In preferred embodiments, the method for producing RNA additionally comprises the step of
(C) Retaining the DNA beads in the reaction vessel and harvesting of the RNA batch.

Accordingly, in preferred embodiments, preferably after performing Step (A) and (B), the DNA beads are retained in the reaction vessel and the RNA batch that has been produced is harvested. The step of retaining the DNA beads may be performed by e.g., using magnetic force, centrifugation, or filtration.

The retaining step facilitates a physical separation of the DNA beads and the produced RNA batch. That physical separation may be required to inter alia re-use the DNA beads for a further IVT cycle (e.g. by feeding fresh IVT buffer to the DNA beads) and/or to remove the DNA beads from the obtained RNA product.

The RNA batch is preferably harvested via an outlet and transferred in an RNA product vessel or bag. In embodiments, the outlet may be a tubing. In embodiments, the harvesting step may be automated and preferably controlled by a pump. In preferred embodiments, the RNA batch is harvested via a tubing that is connected with a product vessel or bag, wherein the transfer of the RNA batch into the product vessel or bag is controlled by a pump.

It is important that the DNA bead are not agglomerated during the retaining and/or harvesting step as DNA bead agglomeration can cause DNA bead escape. Such a DNA bead escape could lead to a contamination of downstream equipment (e.g. purification equipment) and may contaminate the RNA product. A DNA bead agglomeration and/or DNA bead escape can be prevented or reduced by optimizing the RNA in vitro transcription composition as defined herein.

In preferred embodiments of the third aspect, the method for producing RNA comprises the steps of
(A) Providing an RNA in vitro transcription composition comprising DNA beads in an IVT buffer contained in a reaction vessel;
(B) Performing a first or initial IVT cycle by mixing the DNA beads with the IVT buffer to produce an RNA batch;
(C) Retaining the DNA beads in the reaction vessel and harvesting of the RNA batch.

In preferred embodiments, the method for producing RNA comprises at least one further IVT cycle that is preferably performed after Step (C).

For performing at least one further IVT cycle, fresh IVT buffer has to be added to the reaction vessel, preferably to the DNA beads that are retained in the reaction vessel. After the addition of fresh IVT buffer, a new IVT cycle may be performed by mixing (e.g. by using magnetic force) the DNA beads with the freshly added IVT buffer to produce a further RNA batch.

In preferred embodiments, the method for producing RNA additionally comprises the step of
(E) Adding fresh IVT buffer into the reaction vessel that comprises the retained DNA beads.

In that context, the IVT buffer that is added in step (E) comprises the components required for an RNA in vitro transcription on the retained DNA beads. That freshly added IVT buffer is preferably configured to reduce or prevent DNA beads agglomeration, e.g. as defined in the context of the first aspect (e.g. characterized by any one of features F1 to F6). The freshly added IVT buffer does not comprise DNA templates or DNA beads. After the addition of fresh IVT buffer into the reaction vessel, a further IVT cycle may be carried out as defined herein.

Accordingly, in preferred embodiments, the method for producing RNA additionally comprises at least one further IVT cycle that comprises the steps of
(E) Adding fresh IVT buffer into the reaction vessel that comprises the retained DNA beads;
(F) Performing of a further IVT cycle by mixing the DNA beads with the freshly added IVT buffer to produce a further RNA batch;
(G) Retaining the DNA beads in the reaction vessel and harvesting of the further RNA batch;
Step (F) is preferably performed using similar or essentially the same conditions that have been chosen for step (B). Step (G) preferably comprises harvesting of the RNA batch via an outlet and transferred in an RNA product vessel or bag.

Suitably, the method for producing RNA as defined herein comprises more than one further IVT cycle as defined herein, for example, 1, 2, 3, 4, 5, 6, 7, 8, 9, 10, 11, 12, 13, 14, 15, 16, 17, 18, 19, or 20 further IVT cycles.

Accordingly, in preferred embodiments, the method additionally comprises at least one further IVT cycle as defined herein, wherein the at least one further IVT cycle is performed 1 to 20 times (e.g. 3 to 10 times), or in other words, wherein the steps (E) to (G) are repeated 1 to 20 times (e.g. 3 to 10 times).

Suitably, the method for producing RNA may comprise a first IVT cycle (or a first round of RNA in vitro transcription) as defined herein, preferably comprising steps (A), (B) and (C) and at least one further IVT cycle (or at least one further round of RNA in vitro transcription) comprising steps (E), (F) and (G).

In the context of the invention, the RNA in vitro transcription composition of the first aspect inter alia solves the as yet undescribed problem of DNA beads agglomeration. In particular, that problem has been observed by the inventors for RNA production methods that comprise more than one IVT cycle using (the same) DNA beads. As shown by the inventors, the risk of DNA beads agglomeration for such methods can be further reduced by implementing a DNA beads washing step or a DNA beads recovery step. Without whishing to be bound to theory, such a wash step may remove molecules such as RNA, spermidine, or Mg²⁺ that may be attached to the DNA beads. By a washing step under certain conditions, these attached molecules can be removed which may prevent or reduce the risk of DNA beads agglomeration.

Accordingly, in preferred embodiments, the method comprises a step of
(D) Washing of the retained DNA beads in a wash buffer, wherein step (D) is preferably performed after step (C) and/or after step (G).

Preferably, during step (D) the DNA beads are mixed (to e.g. keep them free floating). Suitably, the mixing is performed by magnetic force.

In embodiments, step (D) comprises at least two washing steps, wherein fresh wash buffer is added to the DNA beads in each of the at least two washing steps.

In preferred embodiments, the wash buffer used in step (D) comprises at least one chelating agent. Preferred in the context of the invention are cation chelating agents, e.g. chelating agents that can bind Mg²⁺ ions.

In preferred embodiments in that context, the chelating agent is EDTA, or EDTA derivatives.

Preferably, the wash buffer comprises the chelating agent, preferably EDTA, at a concentration ranging from 0.1 mM to 50 mM, preferably ranging from 1 mM to 10 mM, more preferably ranging from 1 mM to 5 mM.

In other embodiments, the wash buffer comprises urea, preferably 0.5M to 5M urea. In that context, the wash buffer may comprise a chelating agent as defined herein and urea as defined herein.

In preferred embodiments in that context, the wash buffer is adjusted to a pH value of between 7.5 and 8.5. In preferred embodiments, the wash buffer has a pH value of about 8.0.

In preferred embodiments in that context, the wash buffer comprises at least one buffering agent. A preferred buffering agent in that context is Tris.

In embodiments in that context, the wash buffer comprises 1 mM to 500 mM buffering agent, preferably Tris. In preferred embodiments, the wash buffer comprises 1 mM to 50 mM buffering agent, preferably Tris.

In preferred embodiments in that context, the wash buffer comprises 1 mM EDTA and 10 mM Tris (pH 8.0).

In preferred embodiments in that context, step (D) is carried out subsequentially with 1-5 reaction volumes. Preferably, step (D) is carried out with at least 2 subsequent reaction volumes. The DNA beads are preferably mixed during the washing step. In embodiments, the washing step is performed for at least 15 minutes. In embodiments, the washing step is performed at a temperature of about 37° C. After the washing step, the DNA beads are retained (e.g., by magnetic force, filtration, centrifugation) and the wash buffer is removed from the reaction vessel (e.g., via a waste port).

In preferred embodiments, step (D) reduces or prevents agglomeration of the DNA beads and/or reduces or prevents attachment of RNA to the DNA beads (during the course of RNA in vitro transcriptions). Furthermore, the implementation of step (D) stabilizes the amount of RNA that is produced in each IVT cycle (e.g., the RNA yield does not decrease from cycle to cycle).

Accordingly, in preferred embodiments, the RNA yield produced in each IVT cycle is essentially stable, in other words, the RNA yield does not decrease from IVT cycle to IVT cycle.

In embodiments, the IVT cycles are performed until an RNA concentration of at least 5 mg/ml is reached. Accordingly, in preferred embodiments, the method for producing RNA comprises the following steps
(A) Providing an RNA in vitro transcription composition as defined herein comprising DNA beads in an IVT buffer contained in a reaction vessel as defined herein;
(B) Performing an IVT cycle by mixing the DNA beads with the IVT buffer to produce an RNA batch;
(C) Retaining the DNA beads in the reaction vessel and harvesting of the RNA batch,
(D) Washing of the retained DNA beads in a wash buffer as defined herein;
(E) Adding fresh IVT buffer into the reaction vessel that comprises the retained DNA beads;
(F) Performing of a further IVT cycle by mixing the DNA beads with the freshly added IVT buffer to produce a further RNA batch;
(G) Retaining the DNA beads in the reaction vessel and harvesting of the further RNA batch;
   and, optionally followed by n further IVT cycles, wherein a further IVT cycle comprises steps (D) to (G).

The method is suitably performed in the following sequence: A, B, C, [D, E, F, G]n, wherein n relates to the number of further IVT cycles (typically n=1 to 20, preferably 3 to 10).

In preferred embodiments, the method can comprises at least one NTP feed step that is preferably performed during the first IVT cycle (step (B)) and/or during any further IVT cycle (step (F)).

An NTP feed step in the context of the invention comprises the addition of NTP feed mixture into the reaction vessel.

In preferred embodiments, the NTP feed is performed 1 to 10 times during the course of each IVT cycle, preferably 2 to 8 times, more preferably 3 to 5 times. In preferred embodiments, the NTP feed mixture does not comprise a cap analogue. Preferably, the NTP feed is performed at predefined intervals or at a timepoint when the NTPs concentration in the reaction is below a certain predefined threshold (e.g. by measuring the course of RNA in vitro transcription).

In preferred embodiments in that context, the NTP feed mixture comprises an NTP mixture that contains G, C, A and U nucleotides. In other preferred embodiments in that context, the NTP feed comprises an NTP mixture that contains G, C, A and m1ψ. In alternative embodiments in that context, the NTP feed mixture comprises an NTP mixture that contains G, C, A and ψ. In preferred embodiments, the NTP feed is optimized for the given RNA sequence to be produced (according to claims 1 to 35 of WO2015188933). Accordingly, for producing an RNA sequence with G:C:A:U of 1:2:3:2, the respective sequence optimized NTP mixture comprises G:C:A:U in a molar ratio of 1:2:3:2.

By implementing NTP feed steps during the course of IVT, the overall RNA yield of an RNA batch can be increased. Moreover, implementing NTP feed steps may be suitable to ensure a more efficient IVT reaction. Moreover, by implementing NTP feed steps, the initial concentration of Mg²⁺ can be kept low (e.g. below 20 mM, below 10 mM as defined herein) which has the advantageous effect that agglomeration of DNA beads can be reduced and/or prevented. Moreover, by implementing NTP feed steps, the formation of by-products such as dsRNA can be reduced or prevented.

In embodiments, the method comprises further optional steps to modify the produced RNA. Preferably, said further optional steps to modify the produced RNA are performed after the harvesting step(s). In one embodiment, the produced RNA is capped in an enzymatic capping step (e.g. by using capping enzymes or immobilized capping enzymes as described in WO2016193226). In one embodiment, the produced RNA is polyadenylated in an enzymatic polyadenylation step (e.g. by using poly(A) polymerase enzymes or immobilized poly(A) polymerase enzymes as described in WO2016174271).

In preferred embodiments, the harvested RNA batch comprises less than 1 μg/ml DNA beads, preferably less than 100 ng/ml DNA beads, more preferably less than 1 ng/ml DNA beads, most preferably the harvested RNA batch is essentially free of DNA beads. In that context, the “harvested RNA batch” relates to the RNA that is directly obtained by the RNA in vitro transcription without performing a step of RNA purification or DNA digestion.

In preferred embodiments, the harvested RNA batch comprises less than 1 μg/ml DNA templates, preferably less than 100 ng/ml DNA templates, more preferably less than 1 ng/ml DNA templates, most preferably the harvested RNA batch is essentially free of DNA templates. In that context, the “harvested RNA batch” relates to the RNA that is directly obtained by the RNA in vitro transcription without performing a step of RNA purification or DNA digestion.

In particularly preferred embodiments, the harvested RNA is essentially free of DNA beads or fragments thereof.

In preferred embodiments, the harvested RNA batch comprises RNA at a concentration of more than 500 mg/L, preferably at RNA concentrations of more than 1 g/L, more preferably at RNA concentrations of more than 5 g/L.

In preferred embodiments, during the course of any of the steps A, B, C, D, E, F, G, less than 10% of the DNA beads contained in the composition are agglomerated, preferably less than 5% of the DNA beads contained in the composition are agglomerated, more preferably less than 1% of the DNA beads contained in the composition are agglomerated. Agglomeration of beads can be determined and quantified by microscopy.

An important feature of the present invention is the performance of an RNA in vitro transcription on DNA that has been immobilized on beads (herein referred to as “DNA beads”). As specified herein, the beads on which the DNA template is immobilized typically comprise a magnetic or (super) paramagnetic material. The use of magnetic or (super) paramagnetic beads in the context of the invention is preferred as a mixing and/or capturing of DNA beads can be easily achieved with magnetic forces, e.g. using a magnet or magnet unit.

A magnet unit may be configured to capture or to introduce a movement of the DNA magnetic beads contained in the reaction vessel. With such movement, a mixing or stirring of DNA magnetic beads and the IVT composition can be induced by the magnetic unit. In case the DNA magnetic beads and the IVT composition are mixed or stirred due to a movement of the DNA magnetic beads induced by the magnetic unit, the thereby established homogeneous mixture of DNA magnetic beads and the IVT composition supports the RNA in vitro transcription of template DNA into RNA. Moreover, after completion of the RNA in vitro transcription, the DNA magnetic beads can be captured or retained by means of the magnet unit.

In an embodiment, the movement of the DNA magnetic beads during the course of IVT (in step B or F) is configured such that a sedimentation of the beads hold in the reaction vessel is avoided. Additionally or alternatively, the movement of the DNA magnetic beads during the course of IVT (in step B or F) is configured to keep the DNA beads comprised on the reaction vessel free-floating in such a way that a sedimentation at the reaction vessel's bottom can be prevented. Further, a mixing or swirling process is improved by keeping the DNA beads during the course of IVT (in step B or F) in the vessel free-floating and/or that coagulation of beads is prevented or reduced. Advantageously, keeping DNA beads free floating and/or avoiding sedimentation of DNA beads reduces the risk of DNA bead agglomeration.

In embodiments, the magnet unit is configured to rotate around a longitudinal axis of the reaction vessel, wherein a rotation direction of the magnet unit is switchable during mixing. The magnet unit may introduce a movement of the DNA magnetic beads in a radial direction of the reaction vessel by inducing the DNA magnetic beads in a radial direction relative to the longitudinal axis of the reaction vessel. The magnetic force can be static or dynamically generated by rotating the magnet unit around the reaction vessel to cause a rotation, accordingly mixing of the DNA magnetic beads. Rotation direction of the magnet unit may be clockwise or anticlockwise relative to the longitudinal axis of the reaction vessel and/or alternately changed.

Accordingly, the DNA magnetic beads may stay free floating in a contactless manner, hence mixing of the components may be improved. As soon as the rotation of the magnet unit stops, the DNA magnetic beads are captured at the inner surface of the reaction vessel and do not rotate any more. Accordingly, the magnet unit is configured to (i) rotate around a longitudinal axis of the reaction vessel to introduce a movement of the DNA magnetic beads as explained above and configured to (ii) capture the DNA magnetic beads when stopping rotation (e.g., in steps B or G).

Accordingly, in preferred embodiments, the mixing of the DNA beads in steps (B) and/or (F) and/or (D) is performed by means of a magnet unit as defined herein. Suitably, the mixing keeps the DNA magnetic beads free-floating during the course of IVT.

In preferred embodiments, the retaining of the DNA beads in steps (C) and/or (G) and/or (D) is performed by means of a magnet unit as defined herein. Suitably, the retaining step keeps the DNA magnetic beads attached to the reaction vessel, preferably to the inner surface of the reaction vessel.

In embodiments, the magnet unit may be an array of electromagnets, a permanent magnet, an electromagnet, or an induction coil. Preferably the magnet unit is positioned in proximity to the outer surface of the reaction vessel. In preferred embodiments, the magnet unit is not in direct contact with the RNA in vitro transcription composition,

In embodiment, the magnet unit comprises a magnetic ring, wherein the magnetic ring is designed to surround the reaction vessel. To facilitate assembling and rotating of the magnet unit around the reaction vessel, the magnet unit may be formed in a ring shape. In other words, the reaction vessel may be positioned in a centre of a ring-shaped magnet unit such that the magnet unit encircles the reaction vessel.

Accordingly, in preferred embodiments, the magnet unit is a magnet ring that encircles the reaction vessel.

To effectively capture or retain DNA magnetic beads, rotation of the magnetic ring may be stopped after mixing the components in the reaction vessel (e.g., in steps B or G).

In an embodiment, the reaction vessel used in the method is paramagnetic such that DNA magnetic beads may be withhold on the inner reaction vessel wall by a cooperation of the paramagnetic vessel and the magnet unit positioned at the reaction vessel. Thereby, the whole reaction vessel may be paramagnetic, or the inner surface of the reaction vessel may be paramagnetic, e.g. by comprising a paramagnetic material or a magnetically conductive material. The term “magnetisable” denotes throughout the invention that the reaction vessel or its inner surface may be temporarily magnetized such that magnetic beads may be attracted and withhold at the reaction vessel wall. The magnetization of the reaction vessel or its inner surface may however be reversed, such that DNA magnetic beads withhold at the reaction vessel wall may be released. It is therefore important that the material of the reaction vessel (e.g. the bioreactor) and/or the inner surface of the reaction vessel (e.g. bioreactor) are not permanently magnetized by switching on the magnet unit (that is, not ferromagnetic).

Accordingly, in a preferred embodiment, the reaction vessel is paramagnetic. In other embodiments, the reaction vessel is configured to allow penetration of a magnetic field without being magnetisable.

Suitably, the method for producing RNA is performed in a bioreactor as described in WO2020002598, preferably as described in claims 1 to 59 of WO2020002598. Particularly suitable bioreactors in the context of the invention are illustrated in FIGS. 1 to 11 of WO2020002598.

In preferred embodiments, the providing step (A) comprises a DNA production step (A1) and a DNA immobilization step (A2) and an optional on-bead DNA trimming step (A3).

In preferred embodiments, the DNA production (A1) step is polymerase chain reaction (PCR). Suitably, a DNA template is amplified using primers to obtain PCR amplified template DNA. Suitably, biotinylated primers are used for PCR amplification to allow an immobilization of the produced DNA template on streptavidin functionalized beads. The produced PCR amplified biotinylated DNA template is preferably purified.

Alternatively, the DNA production (A1) step is bacterial plasmid DNA (pDNA) amplification. Suitably, a DNA template is amplified using bacterial fermentation. Suitably, the produced pDNA is linearized and functionalized with biotin to allow an immobilization of the produced DNA template on streptavidin functionalized beads. The produced biotinylated DNA template is preferably purified.

In preferred embodiments, the DNA immobilization step (A2) comprises a step of combining biotinylated DNA (e.g. PCR template or linear pDNA) with a streptavidin functionalized bead (e.g. magnetic bead) in an immobilization buffer. Step A2 may be performed in the reaction vessel that is used for the RNA production method. After immobilization, the obtained DNA beads may be further modified.

A preferred modification of DNA beads comprise an on-bead DNA trimming step (A3), for example in case where the DNA have been produced by PCR amplification.

DNA templates for RNA in vitro transcription may comprise a Poly(A) cassette at the terminus of the DNA.

That poly(A) cassette can be introduced via the PCR primers or may be amplified using flanking primers. The amplification of the poly(A) cassette via flanking primers has the advantage that the PCR reaction is more homogeneous and more stable (e.g. constant size of PCR products). A disadvantage is that RNA molecules that are produced using such a DNA template would not terminate with a poly(A) cassette. However, it has been shown that it is important to for biological activity for e.g. mRNAs to have a terminal poly(A)sequence (that is, the poly(A) cassette is the terminus of the RNA; see e.g., WO2022162027).

Accordingly, in embodiments where PCR is used to amplify a poly(A) comprising DNA template in step A1 via flanking primers, the immobilized DNA template (obtained after step A2) may additionally be trimmed using restriction endonucleases that bind on the flanking region of the DNA and cleave in or at the poly(A) sequence to generate a poly(A) 3′ end. Suitably, TypIIS restriction enzymes are used in step A3 to carry out an on-bead DNA digestion to obtain a poly(A)sequence 3′ end of the DNA template. The result of the DNA trimming step A3 is a bead that comprises an immobilized DNA template with a free non-immobilized poly(A) 3′ end.

In preferred embodiments, the method additionally comprises a step of RNA purification of the harvested RNA to obtain a purified RNA.

The term “purified RNA” or “purified mRNA” as used herein has to be understood as RNA which has a higher purity after certain purification steps (e.g. HPLC, TFF, oligo(dT) purification, precipitation steps) than the starting material (e.g. harvested in vitro transcribed RNA). Typical impurities that are essentially not present in purified RNA comprise peptides or proteins (e.g. enzymes derived from DNA dependent RNA in vitro transcription, e.g. RNA polymerases, RNases, pyrophosphatase, restriction endonuclease, DNase), spermidine, BSA, abortive RNA sequences, RNA fragments (short double stranded RNA (dsRNA)), free nucleotides (modified nucleotides, conventional NTPs, cap analogue), template DNA fragments, buffer components (HEPES, TRIS, MgCl₂), DNA beads, etc. Other potential impurities that may be derived from e.g. fermentation procedures comprise bacterial impurities (bioburden, bacterial DNA) or impurities derived from purification procedures (organic solvents etc.). Accordingly, it is desirable in this regard for the “degree of RNA purity” to be as close as possible to 100%. Accordingly, “purified RNA” as used herein has a degree of purity of more than 75%, 80%, 85%, very particularly 90%, 91%, 92%, 93%, 94%, 95%, 96%, 97%, 98% and most favourably 99% or more. The degree of purity is e.g. determined by an analytical HPLC, wherein the percentages provided above correspond to the ratio between the area of the peak for the target RNA and the total area of all peaks including the peaks representing the by-products. Alternatively, the degree of purity is e.g. determined by an analytical agarose gel electrophoresis or capillary gel electrophoresis.

Suitably, the harvested RNA is transferred to an RNA purification module and subjected to at least one purification step.

In preferred embodiments, the RNA purification step (H) comprises at least one step selected from RP-HPLC, AEX, SEC, hydroxyapatite chromatography, TFF, filtration, precipitation, core-bead flow through chromatography, oligo(dT) purification, cellulose-based purification, or any combination thereof.

In more preferred embodiments, the RNA purification step (H) comprises at least step selected from RP-HPLC (preferably performed as described in WO2008077592), TFF (preferably performed as described in WO2016193206). In particularly preferred embodiments, the RNA purification step (H) comprises at least one step of oligo(dT) purification, and, optionally, at least one step of TFF and/or RP-HPLC. In other preferred embodiments, the RNA purification step (H) comprises at least one step of oligo(dT) purification and at least one step of cellulose purification (preferably performed as described in WO2022162027).

In preferred embodiments, the method of producing RNA does not comprise a DNAse treatment step. Suitably, the DNA beads of the method are captured and retained effectively so that a DNA contamination of the produced RNA is prevented. That DNA beads capturing allows for a re-use of the DNA beads and makes a DNAse treatment step obsolete. As DNAse treatment can introduces further contaminations into the produced RNA batch (e.g. DNA fragments, DNAse enzyme), the fact that DNA beads are used improves the purity of the produced RNA.

In preferred embodiments, the method additionally comprises a step (I) of formulating the RNA, preferably formulating the purified RNA obtained from step (H).

In preferred embodiments, step (I) is an LNP formulation step. For example, LNPs may be formulated by mixing a lipid composition with an aqueous RNA composition via a T-piece or Y-piece mixing element.

Alternatively, LNPs may be formulated using microfluidic mixing. After LNP formulation, the LNPs may be purified or re-buffered using filtration and/or TFF.

In preferred embodiments, the method additionally comprises a fill and finish step (J) for e.g. obtaining a formulated RNA medicament. Suitably, the formulated RNA medicament, preferably the LNP formulated RNA medicament, is filled aseptically in liquid or lyophilized form. Accordingly, a lyophilization step (preferably carried out according to WO2016165831), a spray-freeze drying step (preferably carried out according to WO2016184576), or a spray drying step (preferably carried out according to WO2016184575) may performed in step (J). Suitably, the medicament is filled into vials or into syringes, preferably syringes that are compatible with LNP formulated RNA medicaments (suitably selected from syringes described in WO2022207862).

Notably, it is advantageous and particularly preferred in the context of the invention that a DNA bead agglomeration is strongly reduced or prevented during the course of IVT to ensure that the downstream method steps (e.g. (H), (I), (J) are not impaired, e.g. by contaminations caused by DNA beads.

Preferably, the method for producing RNA produces more than 1 mg of RNA, more than 1 g of RNA, more than 10 g of RNA, more than 100 g of RNA. For example 1 mg to 1000 g of RNA, preferably 1 mg to 100 g of RNA, more preferably 1 g to 100 g of RNA. That RNA is optionally formulated in lipid-based carriers, e.g. in LNPs as defined herein.

Preferably, the produced RNA has an RNA purity of at least 85%, preferably of at least 90%, more preferably of at least 95%, 96%, 97%, 98%, or 99%. Preferably, the produced RNA has an RNA integrity of at least 75%, preferably of at least 80%, more preferably of at least 85%. RNA integrity is determined using IP-RP-HPLC.

Preferably, the method is an RNA production method that can be operated under GMP. Preferably, the method is an RNA production method that is configured and optimized to be used in an automated RNA manufacturing device (see fourth aspect).

### RNA Manufacturing Device:

In a fourth aspect, the present invention provides an RNA manufacturing device that comprises the RNA in vitro transcription composition of the first aspect, or that is configured to perform the RNA production method of the third aspect.

It has to be noted that features and embodiments that are described in the context of the RNA in vitro transcription composition of the first aspect or the method of the third aspect may also be applicable to the RNA manufacturing device of the fourth aspect. Likewise, features and embodiments that are described in the context of the RNA manufacturing device of the fourth aspect may also be applicable to the RNA in vitro transcription composition of the first aspect or the method of the third aspect.

In preferred embodiments, the RNA manufacturing device comprises a bioreactor for RNA in vitro transcription comprising:
(a) a reaction vessel suitable to hold the RNA in vitro transcription composition of the first aspect,
wherein the DNA beads are DNA templates immobilized on magnetic beads, and
(b) a magnet unit positioned at the reaction vessel,
wherein the magnet unit is configured to capture or to introduce a movement of the DNA beads.

Preferably, the magnet unit is a permanent magnet or an electromagnet movable in a longitudinal direction along at least one of a longitudinal axis of the reaction vessel, such that the magnet unit is configure to capture or to introduce a movement into the DNA beads

In preferred embodiments of the RNA manufacturing device, the magnet unit is a magnet ring that encircles the reaction vessel.

In particularly preferred embodiments, the RNA manufacturing device comprises a bioreactor as described in WO2020002598, preferably as described in claims 1 to 59 of WO2020002598. Particularly suitable bioreactors in the context of the invention are illustrated in FIGS. 1 to 11 of WO2020002598.

In embodiments, the RNA manufacturing device additionally comprises a module for DNA production (e.g. for performing PCR), suitably a module as described in WO2022112498, in particular as described in claims 1 to 28 of WO2022112498, or as illustrated in FIGS. 1 to 11 of WO2022112498.

In embodiments, the RNA manufacturing device additionally comprises a purification module, preferably configured to perform purification of RNA using RP-HPLC, AEX, SEC, hydroxyapatite chromatography, TFF, filtration, precipitation, core-bead flow through chromatography, oligo(dT) purification, cellulose-based purification, or any combination thereof.

In embodiments, the RNA manufacturing device additionally comprises a formulation module, preferably an LNP formulation module and/or a fill and finish module suitable for aseptic filling of an RNA medicament.

In preferred embodiments, the bioreactor for RNA in vitro transcription and further optional modules are integrated into a GMP manufacturing device, module, or system. Preferably, the GMP manufacturing device, module, or system is a device, module, or system as described in WO2022049093, in particular as described in claims 1 to 72 of WO2022049093, or as illustrated in FIGS. 1 to 6 of WO2022049093.

### Use of on an IVT Buffer to Prevent or Reduce Agglomeration of DNA Beads:

In a further aspect, the invention relates to the use of an IVT buffer for preventing or reducing agglomeration of DNA beads (and hence also RNA yield), preferably for preventing or reducing agglomeration of DNA beads in an RNA manufacturing process during the course of RNA in vitro transcription.

It has to be noted that features and embodiments that are described in the context of the RNA in vitro transcription composition of the first aspect or the method of the third aspect may also be applicable to the present aspect.

Accordingly, the IVT buffer of the use is characterized by any of the features that relate to suitable IVT buffers as disclosed herein (e.g. as disclosed in the first aspect). For example, features relating to preferred amounts of spermidine, DTT, NTPs, Mg²⁺, Mg²⁺ to NTP ratios, RNA polymerase, or other additives as defined herein may likewise apply to the IVT buffer of the use of the present aspect.

Particularly preferred embodiments of the IVT buffer of the present aspect are summarized in the following.

Suitably, the IVT buffer comprises less than 2 mM spermidine and Mg²⁺ to NTP in a molar ratio of below 1.4. Preferably, the IVT buffer comprises less than 25 mM Mg²⁺.

Preferably, the IVT buffer of the use is characterized by at least one, at least two, or a combination of the following features F1 to F6
F1: less than 1 mM spermidine, preferably free of spermidine;
F2: Mg²⁺ at a concentration between 1 mM to 25 mM, preferably less than 20 mM Mg²⁺;
F3: the added total concentration of spermidine and Mg²⁺ in the IVT buffer is less than 30 mM;
F4: the molar ratio of Mg²⁺ to NTPs is ranging from 1.2 to 0.5, preferably ranging from 0.8 to 0.6;
F5: NTPs at a total concentration of at least 3 mM, preferably ranging from 3 mM to 35 mM;
F6: less than 10 mM DTT, preferably less than or about 1 mM DTT;

In preferred embodiments in that context, the IVT buffer of the use is characterized by the following features, sorted by increasing preference:
F1 and F2
F1 and F2 and F3
F1 and F2 and F4
F3 and F4
F1 and F2 and F3 and F4
F3 and F5 or F2 and F5
F1 and F2 and F3 and F4 and F5
F1 and F2 and F3 and F4 and F5 and F6

In preferred embodiments of the use, the DNA beads may be characterized by any one of the features disclosed in the context of the first of third aspect.

According to preferred embodiments, the DNA beads of the use are characterized by at least one, at least two, or a combination of the following features B1 to B5
B1: comprise a magnetizable material, preferably a (super) paramagnetic material;
B2: diameter in a range of 1 μm to 10 μm, preferably in a range of 4 μm to 5 μm, e.g. 4.5 μg;
B3: comprise polystyrene or a derivative of polystyrene;
B4: the bead surface comprises about 1 ng DNA/mm² to about 5 ng DNA/mm²
B5: beads comprise a biotin-streptavidin immobilized DNA template;

In preferred embodiments in that context, the DNA beads of the use are characterized by the following features, sorted by increasing preference:
B1 and B2;
B1 and B2 and B3;
B1 and B2 and B3 and B4;
B1 and B2 and B3 and B4 and B5;

Preferred examples of beads that are suitable in the context of the invention are Dynabeads® such as Dynabeads® M-270, Dynabeads® M-280, Dynabeads® M-450, or Dynabeads® MyOne. In specific embodiments of the method, the beads on which the DNA template is immobilized are Dynabeads® M-450, e.g. Dynabeads® M-450 E beads.

In preferred embodiments of the use, agglomeration of DNA beads is reduced or prevented at RNA concentrations of more than 100 mg/L, preferably at RNA concentrations of more than 1 g/L, more preferably at RNA concentrations of more than 5 g/L.

In preferred embodiments of the use, agglomeration of DNA beads is reduced or prevented if the DNA beads are used for more than one RNA in vitro transcription cycle. For example, agglomeration of DNA beads is reduced or prevented if the DNA beads are used for 1 to 20 IVT cycles, preferably for 3 to 10 IVT cycles.

### Use of a Wash Buffer Comprising a Chelating Agent to Prevent or Reduce Agglomeration of DNA Beads

In a further aspect, the invention relates to the use of a buffer (a wash buffer) comprising a chelating agent to prevent or reduce agglomeration of DNA beads, preferably for preventing or reducing agglomeration of DNA beads in an RNA manufacturing process, e.g. during the course of RNA in vitro transcription.

As shown by the inventors, the risk of DNA beads agglomeration in RNA in vitro transcription can be further reduced by implementing at least one DNA beads wash step or at least one DNA beads recovery step. That wash step is preferably carried out between IVT cycles. Without whishing to be bound to theory, such a wash step may remove molecules such as RNA, spermidine, or Mg²⁺ that may be attached to the DNA beads. By a washing step under certain conditions, these attached molecules can be removed which may prevent or reduce the risk of DNA beads agglomeration.

In preferred embodiments of the use, the wash buffer comprises at least one chelating agent. Preferred in the context of the invention are cation chelating agents, e.g. chelating agents that can bind Mg²⁺ ions.

In preferred embodiments in that context, the chelating agent is EDTA or a derivative of EDTA. Preferably, the wash buffer of the use comprises the chelating agent, preferably EDTA, at a concentration ranging from 0.1 mM to 50 mM, ranging from 1 mM to 10 mM, preferably ranging from 1 mM to 5 mM.

In other embodiments, the wash buffer comprises urea, preferably 0.5M to 5M urea. In that context, the wash buffer may comprise a chelating agent as defined herein and urea as defined herein.

In preferred embodiments of the use, the wash buffer is adjusted to a pH value of between 7.5 and 8.5. In preferred embodiments, the wash buffer has a pH value of about 8.0.

In preferred embodiments of the use, the wash buffer comprises at least one buffering agent. A preferred buffering agent in that context is Tris.

In embodiments of the use, the wash buffer comprises 1 mM to 500 mM buffering agent, preferably Tris. In preferred embodiments, the wash buffer comprises 1 mM to 50 mM buffering agent, preferably Tris.

In preferred embodiments of the use, the wash buffer comprises 1 mM EDTA and 10 mM Tris (pH 8.0).

In preferred embodiments of the use, the wash buffer used between two RNA in vitro transcription cycles as defined herein to reduce DNA beads agglomeration or to recover DNA beads for a further IVT cycle.

In preferred embodiments of the use, the wash buffer is preventing or reducing agglomeration of DNA beads that have certain characteristics. Suitably, the DNA beads are as defined in the context of the first aspect, preferably characterized by at least one or all features B1 to B5. Preferred examples of beads that are suitable that context are Dynabeads® such as Dynabeads® M-270, Dynabeads® M-280, Dynabeads® M-450, or Dynabeads® MyOne. In specific embodiments, the beads are Dynabeads® M-450, e.g. M-450 E.

### BRIEF DESCRIPTION OF THE DRAWINGS

FIG. 1 shows a schematic view of an exemplary bioreactor and a magnet unit as used herein (image adapted from WO2020002598). A magnet unit 3 encircles the reaction vessel 2 that contains the IVT composition 1 such that the magnet unit 3 can rotate around the reaction vessel 2 (e.g. to introduce a mixing of DNA magnetic beads) or can stop (e.g. to capture DNA magnetic beads). The magnet unit 3 is attached to a means 4 that can move the magnet unit 3. Accordingly, a homogeneous mixing of the IVT composition 1 in the reaction vessel 2 is realised by introducing a movement into the DNA magnetic beads. IVT reagents can be added via an inlet port 5 and RNA product can be drained via an outlet port 6.

FIG. 2 shows an overlay of HPLC chromatograms in a cycled RNA in vitro transcription reaction using DNA beads. The RNA yield decreases from cycle to cycle, visible as a reduced area under the curve for each chromatogram. Numbered arrows indicate the chromatogram of the respective IVT cycles 1 to 8. Further details are provided in Example 1.

FIG. 3 shows endoscopic photographs from the inside of the bioreactor (initial IVT and IVT cycles C1 to C11). Dashed circle indicate the position of the outlet port opening. Bead agglomerate or pellets (coloured in black) are indicated by “P”. White arrows in images C9 to C11 indicate that magnetic beads escaped from the bioreactor and aggregate in large amounts at the bottom (dark black areas). Further details are provided in Example 2.

FIG. 4 shows microscopic images of DNA magnetic beads that escaped from the bioreactor after cycle 9 of Example 2. The respective treatments are indicated. Scale bars represent 50 μm. Further details are provided in Example 3.

FIG. 5 shows microscopic photographs of DNA magnetic beads that escaped from the bioreactor after cycle 9 of Example 2. The respective treatments are indicated. Scale bars represent 50 μm. Further details are provided in Example 3.

FIG. 6 shows endoscopic photographs from the inside of the bioreactor for RNA in vitro transcription in the presence of 2 mM spermidine. Image 1 shows end of initial IVT cycle; Image 2 shows end of cycle 1; Image 3 shows inter cycle bead wash with TE after cycle 1; Image 4 shows end of cycle 2; Image 5 shows end of cycle 3. Dashed circle indicate the position of the outlet port opening. Bead agglomerate or bead pellets (coloured in black) are indicated by “P”. White arrows in Panel 3 indicate that magnetic beads escaped from the bioreactor and aggregate in large amounts at the bottom (dark black areas). Further details are provided in Example 5.

FIG. 7 shows endoscopic images from the inside of the bioreactor for RNA in vitro transcription in the absence of spermidine. Image 1 shows end of initial IVT cycle; Image 2 shows end of cycle 1; Image 3 shows inter cycle bead wash with TE after cycle 1; Image 4 shows end of cycle 2; Image 5 shows end of cycle 3. Dashed circle indicate the position of the outlet port opening. Bead agglomerate or bead pellets are not visible. No magnetic beads escaped from the bioreactor or aggregate in large amounts at the bottom (compare with FIG. 6). Further details are provided in Example 5.

FIG. 8 shows RNA yields obtained from cycled RNA IVT reactions to produce RNA R1 in the presence of varying concentrations of spermidine (0 to 6 mM). Further details are provided in Example 6.

### EXAMPLES

In the following, examples illustrating various embodiments and aspects of the invention are presented. However, the present invention shall not to be limited in scope by the specific embodiments presented herein, and should rather be understood as being applicable to other compositions and/or methods as for example defined in the specification. Accordingly, the following preparations and examples are given to enable those skilled in the art to more clearly understand and to practice the present invention. The present invention is not limited in scope by the exemplified embodiments, which are merely intended as illustrations of single aspects of the invention, and methods, which are functionally equivalent, are within the scope of the invention. Indeed, various modifications of the invention in addition to those described herein will become readily apparent to those skilled in the art from the foregoing description, accompanying figures and the examples below.

### Example A: RNA Constructs of the Experiment

### Table 1: RNA Constructs Produced in the Present Examples

In the following Table, features of the used mRNA constructs are described for RNA R1 encoding multiple tumor epitopes, RNA R2 encoding a viral antigen, and RNA R3 encoding a tumor antigen.

TABLE 1
RNA constructs produced in the present Examples

| | RNA | | | | |
|---|---|---|---|---|---|
| ID | Length | Cap | (5′UTR / 3′UTR) | GC content % | 3′ end |
| R1 | 1514 | Cap1 | HSD17B4 / PSMB3 | 60.8 | hSL-A100 |
| R2 | 4009 | Cap1 | — / muag | 62.5 | A64-hSL |
| R3 | 1928 | Cap1 | HSD17B4 / PSMB3 | 61.7 | hSL-A100 |

### Example 1: a Reduced RNA Yield was Observed in Cycled RNA In Vitro Transcription Using DNA Beads

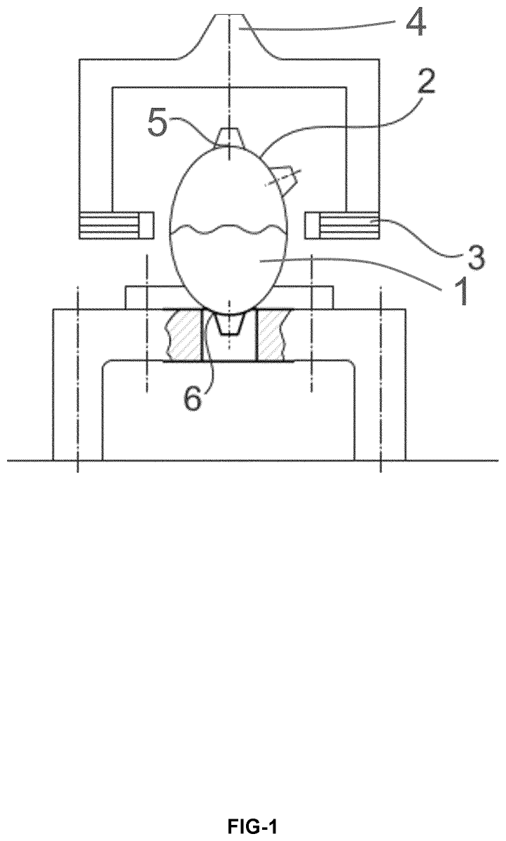

PCR amplified biotinylated linear template DNA was immobilized on streptavidin functionalized magnetic beads (Dynabeads® M-450 E; Thermo Fisher) to produce DNA beads. The obtained DNA magnetic beads were used in a cycled RNA vitro transcription process using an IVT bioreactor (an exemplary setup of the bioreactor is shown in FIG. 1). The produced RNA had a length of 1514 nucleotides (R1; see Table 1).

For the RNA in vitro transcription of the present experiment, the following RNA in vitro transcription composition was used: 34 mg/mL DNA beads (60 ng/μL DNA), 27 mM NTPs (sequence optimized), 19 mM MgCl₂, 80 mM Tris pH 8.0, 2 mM spermidine, 1 mM DTT. Additionally, the RNA in vitro transcription composition comprised T7 RNA polymerase, Cap analog (CleanCap AG), pyrophosphatase and RNAse inhibitor. The IVT reaction was incubated for about 90 minutes at 37° C.

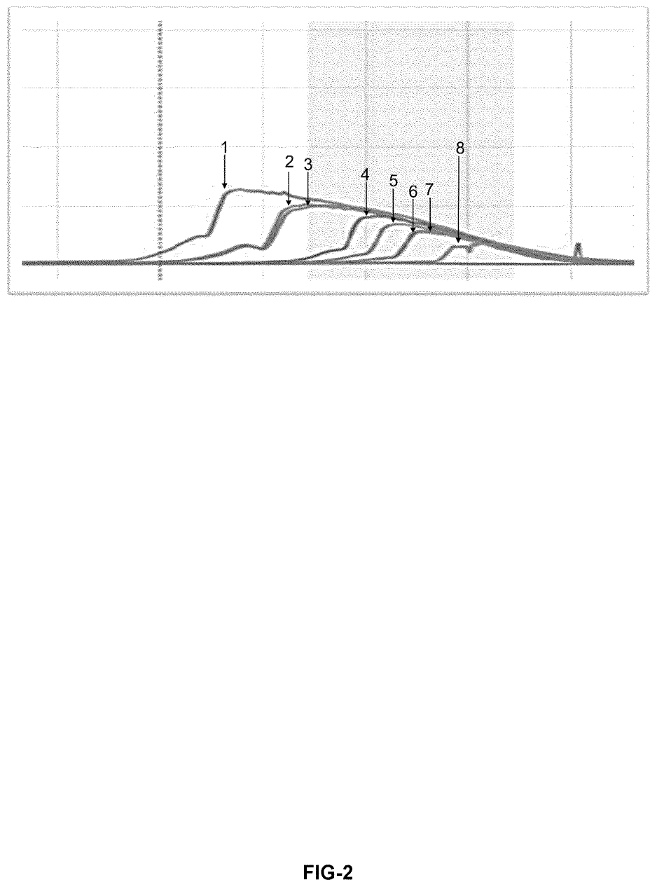

During the course of the IVT, magnets were used to introduce a mixing of the RNA in vitro transcription composition. The IVT process was stopped by capturing the DNA beads and draining the reactor to obtain product RNA. Following that, captured DNA beads were washed 2× with fresh IVT buffer (comprising 80 mM Tris pH 8.0, 2 mM spermidine, 1 mM DTT) in the reactor. Fresh RNA in vitro transcription composition was added to the reactor to initiate the next RNA IVT cycle by re-using the DNA beads. That process was repeated 8 times. Obtained RNA products from each IVT cycle were purified using RP-HPLC. Exemplary HPLC chromatograms are shown as an overlay in FIG. 2.

### Result

During manufacturing of RNA R1, a decrease in RNA yield from the initial cycle (first RNA in vitro transcription; indicated by “1” in chromatogram) to any of the subsequent cycles could be observed (see FIG. 2). The RNA yield continued to decline from cycle to cycle such that e.g. for cycle 8 the RNA yield was strongly reduced (indicated by “8” in chromatogram). No RNA product at all could be eluted from the HPLC column for IVT cycle 9. Another important observation was that DNA beads got lost during the course of the RNA manufacturing process which was caused by DNA bead agglomeration (see following Examples).

### Example 2: a Reduced RNA Yield was Observed in Cycled RNA In Vitro Transcription

PCR amplified biotinylated linear template DNA was immobilized on streptavidin functionalized magnetic beads (Dynabeads® M-450 E; Thermo Fisher) to produce DNA beads. The obtained magnetic DNA beads were used in a cycled RNA vitro transcription process using an IVT bioreactor (exemplary setup of the bioreactor is shown in FIG. 1). The produced RNA had the length of 4009 nucleotides (R2; see Table 1).

For the RNA in vitro transcription of the present experiment, the following RNA in vitro transcription composition was used: 34 mg/mL DNA beads (60 ng/μL DNA), 27 mM NTPs (sequence optimized), 19 mM MgCl₂, 80 mM Tris pH 8.0, 2 mM spermidine, 1 mM DTT. Additionally, the RNA in vitro transcription composition comprised T7 RNA polymerase, Cap analog (CleanCap AG), pyrophosphatase and RNAse inhibitor in typical concentrations. The IVT reaction was incubated for about 90 minutes at 37° C.

During the course of IVT, magnets were used to introduce a mixing of the RNA in vitro transcription composition. The IVT process was stopped by capturing the DNA beads and draining the reactor to obtain product RNA. Following that, captured DNA beads were washed once with buffer A (80 mM Tris pH 8.0, 2 mM spermidine, 1 mM DTT). Fresh RNA in vitro transcription composition was added to the reactor to initiate the next RNA IVT cycle by re-using the DNA beads. In total, 12 IVT cycles were performed.

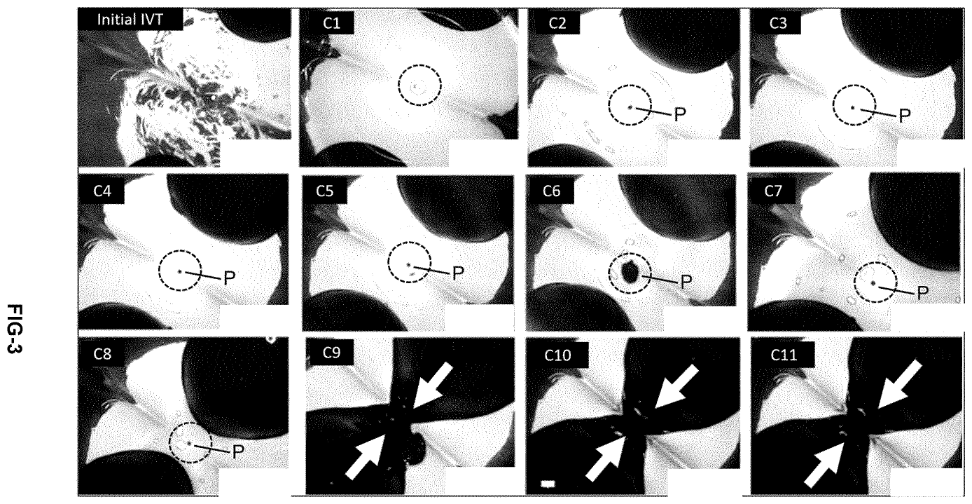

To analyze the behavior of DNA beads during the course of the IVT process in the bioreactor, an endoscope was placed inside the bioreactor to observe the outlet port opening of the bioreactor at the moment of bead separation which happens at the end of an IVT cycle (dashed circles in FIG. 3).

### Result

Using endoscopic imaging, a suitable mixing of the DNA beads was observed after the initial IVT. After that initial IVT, the DNA beads were nicely distributed and did not accumulate around the opening of the bioreactor. A bead agglomerate or pellet (indicated by “P” in FIG. 3) started to form and expand with every additional IVT cycle, and the magnetic beads started to approach the opening on the bottom of the bioreactor (opening is positioned in the centre of the circles in FIG. 3). Additionally, from cycle 5 onwards, larger bead agglomerates could be detected in the endoscope images. For IVT cycle 9, magnetic beads escaped from the bioreactor (indicated by arrows in FIG. 3) and aggregate in large amounts at the bottom. DNA beads that escaped from the bioreactor after cycle 9 were recovered from a depth filter that was positioned after the outlet of the bioreactor, and the DNA beads were further analysed (see Example 3).

The results clearly show that a cycled RNA in vitro transcription using DNA beads under these conditions is associated with fundamental problems, and that the drop in RNA yield that has been observed in Example 1 was caused by DNA bead agglomeration. Accordingly, further adaptations are needed to optimize RNA in vitro transcription using DNA beads.

### Example 3: Microscopic Evaluation of DNA Bead Agglomerates

Magnetic beads that escaped from the bioreactor after cycle 9 of Example 2 were recovered from the depth filter by pushing 1×IVT buffer with a syringe through the filter. Aggregates of magnetic beads were analyzed by a phase-contrast microscope at 1000-fold magnification.

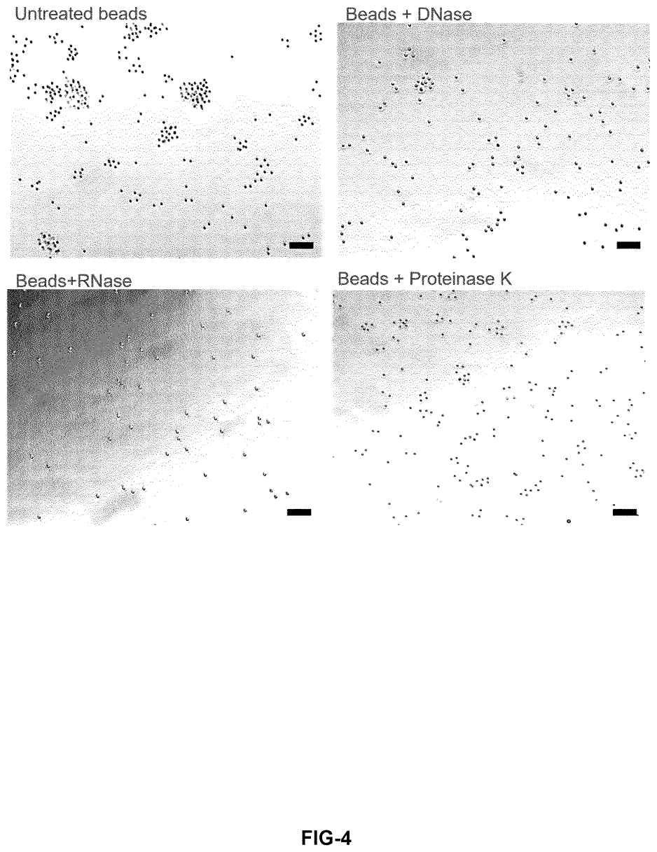

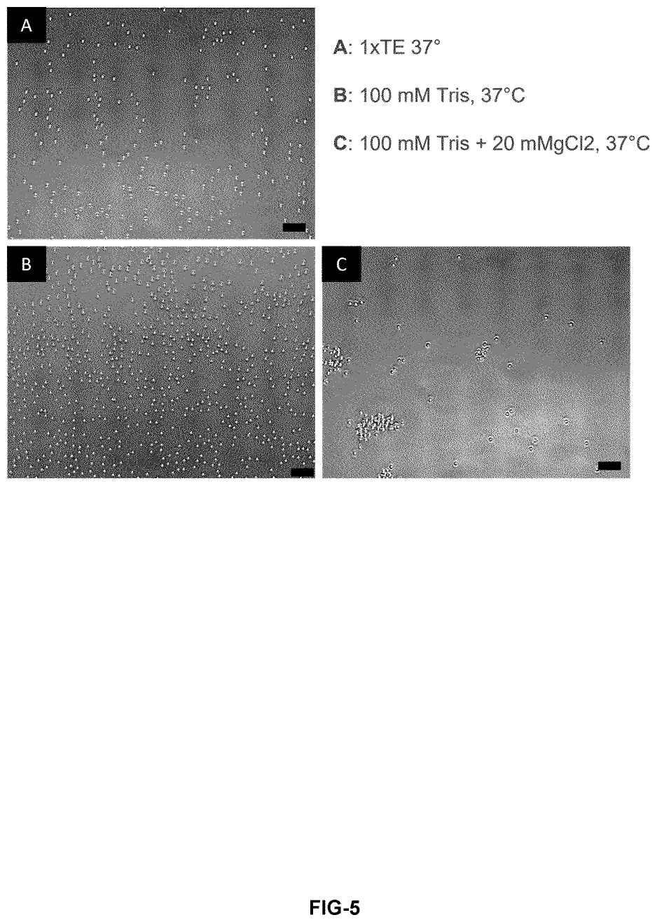

To identify the cause of DNA bead agglomeration, agglomerated DNA magnetic beads of cycle 9 were treated with RNase to digest RNA, DNAse to digest DNA, or Proteinase K to digest proteins (see FIG. 4). Moreover, DNA magnetic beads were treated with different buffers such as 1×TE buffer (10 mM Tris pH 8.0, 1 mM EDTA), 100 mM Tris, and 100 mM Tris+20 mM MgCl₂ (see FIG. 5).

### Result

As shown in FIG. 4, Incubation of the DNA beads with RNase led to dissociation of the DNA bead aggregates while DNase and Proteinase K had no effect. As shown in FIG. 5, incubation in 1×TE at 37° C. for 15 min dissolved the DNA bead aggregates. On the other hand, 20 mM MgCl₂ could stabilize the DNA bead aggregates at 37° C. in 100 mM Tris pH 9 while in the DNA bead aggregates dissolved in the absence of MgCl₂. Thus, the aggregation of the DNA magnetic beads was dependent on RNA that unspecifically bound to the magnetic beads. Moreover, the presence of Mg²⁺ ions strongly stabilized the agglomeration of DNA beads.

In conclusion, reduction of Mg²⁺ ions in the RNA in vitro transcription may further reduce the unwanted DNA bead agglomeration. Also, washing steps in a buffer comprising a chelating agent (e.g. EDTA in Tris) allows to separate DNA bead aggregates formed in the bioreactor (potentially promoted by RNA) during IVT cycles.

### Example 4: DNA Beads Agglomerates are Stabilized by Mg²⁺ in a Model System

To further evaluate the impact of Mg²⁺ on bead agglomeration, and to find potential wash buffers to resolve such bead agglomerates, bead agglomerates were artificially induced in a model system and the effect of different wash buffers was analysed.

30 ml MyOne Streptavidin magnetic beads (Thermo Fisher; 1 μM diameter) or 34 ml M-450 Streptavidin beads (Thermo Fisher; 4.5 μm diameter) were incubated in 0.6 g/L R2 RNA and agglomeration was induced in the presence of 1M NaCl and 10% Peg-8000 in test tubes. To evaluate whether R2 RNA could be recovered from the bead surface by the washing solution, agglomerated beads were washed 3 times for 5 minutes with (i) 1×TE buffer (10 mM Tris pH8.0, 1 mM EDTA), (ii) 1× buffer A (80 mM Tris pH 8.0, 2 mM spermidine, 1 mM DTT), or (iii) 1× buffer A (80 mM Tris pH 8.0, 2 mM spermidine, 1 mM DTT) comprising 19 mM MgCl₂. The results are summarized in Table 2 and Table 3. The RNA concentration in the washing solutions was quantified by UV absorbance using a NanodropOne device.

TABLE 2
Bound RNA on different bead types. The fraction bound
is calculated relative to the RNA concentration used
for precipitation by 1M NaCl and 10% Peg-8000.

| Bead type | % RNA bound on the bead surface |
|---|---|
| MyOne Streptavidine beads 1 μm | 98 |
| M-450 Streptavidine beads 4.5 μm | 23 |

TABLE 3
Recovery of RNA from M-450 E Streptavidine beads by different
washing solutions. Normalization was performed relative
to the RNA concentration recovered by 1xTE

| Wash strategy | Relative RNA recovery [%] |
|---|---|
| 1x TE | 100 |
| 1x buffer A | 104 |
| 1x buffer A + 19 mM MgCl₂ | 1.1 |

### Result

As shown in Table 2, RNA precipitation on the surface of different magnetic beads could be readily achieved by 1 M NaCl and Peg-8000 as molecular crowder in the absence of DNA. Accordingly, the data suggests that attached RNA may play a role in the process of beads agglomeration. This is in accordance with the finding of Example 3 that RNAse treatment leads to dissociation of the DNA bead aggregates while DNAse treatment was not effective. At comparable concentrations, RNA binding was more efficient for MyOne Streptavidin beads with a smaller diameter.

As shown in Table 3, RNA bound to the M-450 E Streptavidin beads was completely dissolved (recovered) when washed in TE buffer or buffer A. However, in the presence of Mg²⁺ ions (1× buffer A+19 mM MgCl₂), bound RNA was stabilized and not dissolved (recovery at 1.1%).

In conclusion, the data supports that a reduction of Mg²⁺ ions in the RNA in vitro transcription may reduce the unwanted DNA bead agglomeration due to RNA binding to the surface. Additionally, washing steps using a buffer comprising a chelating agent (e.g. EDTA in Tris) could be implemented to reduce or prevent bead agglomeration.

### Example 5: Identification of Spermidine as a Further Cause for Bead Agglomeration

PCR amplified biotinylated linear template DNA was immobilized on streptavidin functionalized magnetic beads (Dynabeads® M-450 E; Thermo Fisher) to produce DNA beads. The obtained magnetic DNA beads were used in a cycled RNA vitro transcription process using an IVT bioreactor (exemplary setup of the bioreactor is shown in FIG. 1) in the presence or absence of spermidine. The produced RNA encoded a tumor antigen and had the length of 1928 nucleotides (R3 RNA; see Table 1).

For the RNA in vitro transcription of the present experiment, the following RNA in vitro transcription composition was used: 34 mg/mL DNA beads (60 ng/μL DNA), 27 mM NTPs (sequence optimized), 19 mM MgCl₂, 80 mM Tris pH 8.0, optionally 2 mM spermidine, 1 mM DTT. Additionally, the RNA in vitro transcription composition comprised T7 RNA polymerase, Cap analog (CleanCap AG), pyrophosphatase and RNAse inhibitor in typical concentrations. The IVT reaction was incubated for about 90 minutes at 37° C.

During the course of IVT, magnets were used to introduce a mixing of the RNA in vitro transcription composition. The IVT process was stopped by capturing the DNA beads and draining the reactor to obtain product RNA. Following that, captured DNA beads were washed 2 times in 1×TE buffer (10 mM Tris pH 8.0, 1 mM EDTA). Fresh RNA in vitro transcription composition was added to the reactor to initiate the next RNA IVT cycle by re-using the DNA beads. In total, 4 IVT cycles were performed.

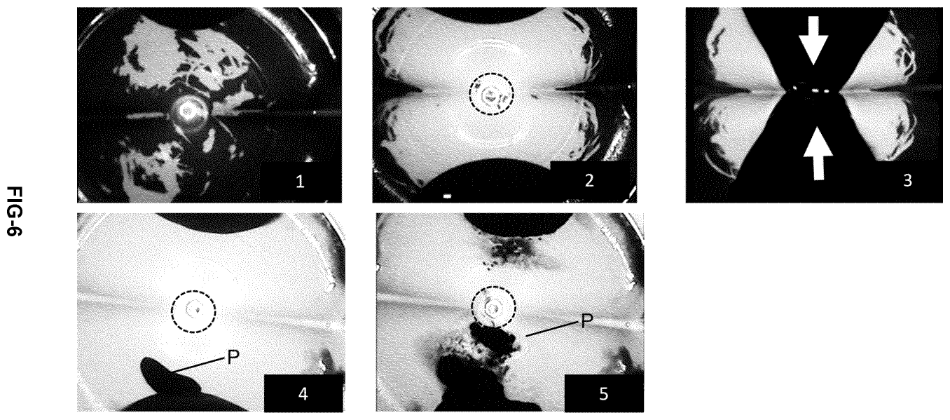

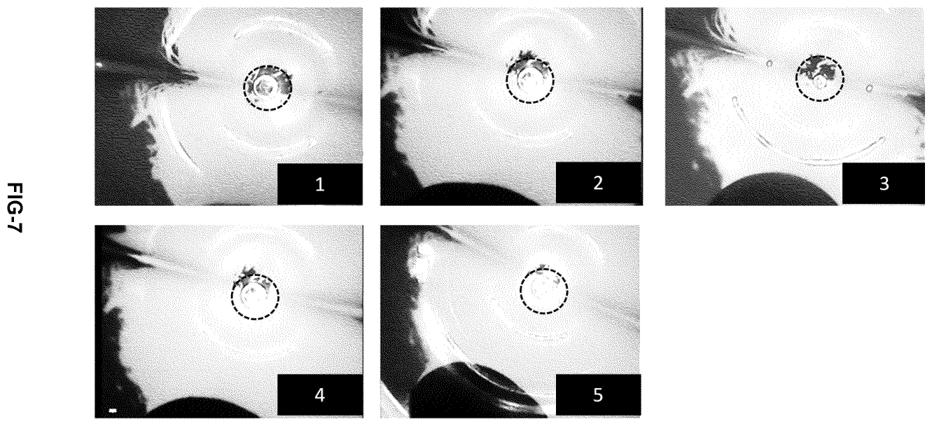

To analyze the behavior of DNA beads during the course of the IVT process in the bioreactor, an endoscope was placed inside the bioreactor to observe the outlet port opening of the bioreactor (dashed circles) in the presence of 2 mM spermidine (see FIG. 6) and in the absence of spermidine (see FIG. 7).

### Result

Using endoscopic imaging, the DNA beads behaviour was analysed after the initial IVT cycle (Image 1 in FIGS. 6 and 7), after the first cycle (Image 1 in FIGS. 6 and 7), after the inter cycle bead wash with TE (Image 2 in FIGS. 6 and 7), at the end of cycle 2 (Image 4 in FIGS. 6 and 7) and at the end of cycle 3 (Image 5 in FIGS. 6 and 7).

As shown in FIG. 6, in the presence of 2 mM spermidine, DNA beads agglomeration or pellet formation (indicated by “P”) was observed. Moreover, after inter cycle bead wash with 1×TE (image 3) magnetic beads escaped from the bioreactor (indicated by arrows). Further bead agglomerate or pellet formation was observed at later cycles (image 4 and 5). As shown in FIG. 7, in the absence of spermidine, bead agglomeration or pellet formation was not observed. Moreover, in the absence of spermidine, DNA magnetic beads did not escape from the bioreactor.

These images clearly show that RNA manufacturing based on cycled RNA in vitro transcription reactions using DNA beads in the absence of spermidine could be performed without bead escape and that DNA beads agglomeration could be prevented.

### Example 6: Spermidine has a Negative Effect on RNA Yield

As shown in previous Examples, spermidine induces bead agglomeration in cycled IVT processes using DNA beads as templates. To evaluate the effect of the spermidine concentration on the RNA yield, cycled IVT reactions were performed to produce RNA R1 in the presence of varying concentrations of spermidine (0-6 mM).

PCR amplified biotinylated linear template DNA was immobilized on streptavidin functionalized magnetic beads (Dynabeads® M-450 E; Thermo Fisher) to produce DNA beads. The obtained magnetic DNA beads were used in a cycled RNA vitro transcription process in reaction tubes in the presence of varying spermidine concentrations.

For the RNA in vitro transcription of the present experiment, the following RNA in vitro transcription composition was used: 34 mg/mL DNA beads (60 ng/μL DNA), 27 mM NTPs (sequence optimized), 19 mM MgCl₂, 80 mM Tris pH 8.0, 1 mM DTT, wherein the spermidine concentration was set to a range from 0 mM spermidine to 6 mM spermidine. Additionally, the RNA in vitro transcription composition comprised T7 RNA polymerase, Cap analog (CleanCap AG), pyrophosphatase and RNAse inhibitor in typical concentrations.

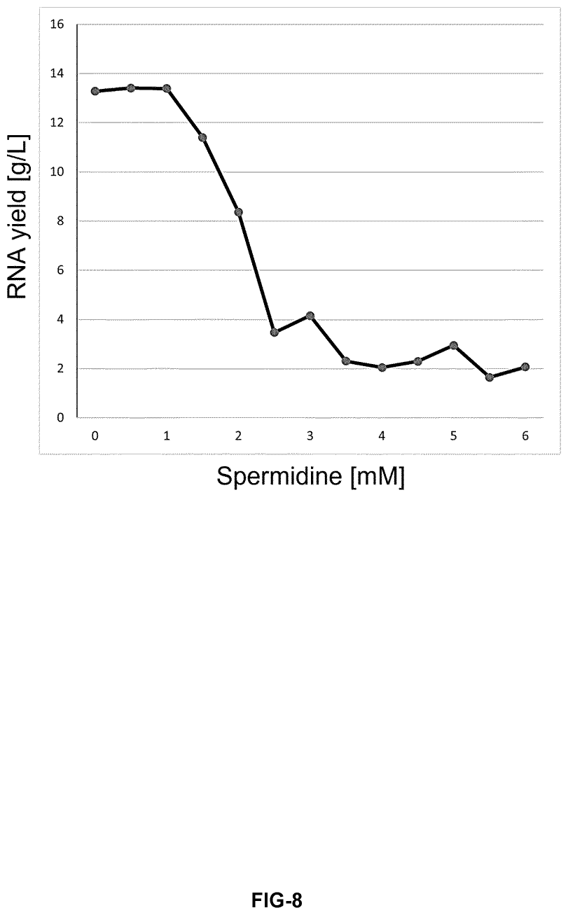

The IVT was performed in 1.5 mL reaction tubes and incubated for about 90 minutes at 37° C. Two IVT cycles were preformed and the RNA concentration for the second IVT cycle was determined by capillary gel electrophoresis. The results are shown in FIG. 8.

### Result

As shown in FIG. 8, increasing concentrations of spermidine had a negative impact on the RNA yield from the RNA in vitro transcription reaction using DNA beads. At concentrations of 2 mM spermidine or more, the RNA yield drops dramatically, most likely because DNA beads started to agglomerate (as observed in previous experiments). A concentration of spermidine of less than about 1.5 mM led to the highest RNA yield.

### Example 7: Effect of Reduced Spermidine Concentrations for IVT Using DNA Beads

As shown in previous Examples, spermidine induces bead agglomeration in cycled IVT processes using DNA beads as templates and—as a consequence—reduces RNA yield. To further evaluate the effect of reduced spermidine on RNA integrity, and capping efficiency, IVT reactions were performed to produce different RNA constructs (R1 and R3) in the presence of varying concentrations of spermidine (0-2 mM).

PCR amplified biotinylated linear template DNA was immobilized on streptavidin functionalized magnetic beads (Dynabeads® M-450 E; Thermo Fisher) to produce DNA beads. The obtained magnetic DNA beads were used in a cycled RNA vitro transcription process in reaction tubes in the presence of varying spermidine concentrations.

For the RNA in vitro transcription of the present experiment, the following RNA in vitro transcription composition was used: 34 mg/mL DNA beads (60 ng/μL DNA), 27 mM NTPs (sequence optimized), 19 mM MgCl₂, 80 mM Tris pH 8.0, 1 mM DTT, wherein the spermidine concentration was set to a range from 0 mM spermidine to 2 mM spermidine. Additionally, the RNA in vitro transcription composition comprised T7 RNA polymerase, Cap analog (CleanCap AG), pyrophosphatase and RNAse inhibitor in typical concentrations.

The IVT was performed in 1.5 mL reaction tubes and incubated for about 90 minutes at 37° C. 2 IVT cycles were preformed and the RNA yield (determined by Qubit BR RNA assay) and the RNA integrity (determined by analytical IP-RP-HPLC) of the non-purified RNA was determined. Further, the capping degree was analysed using a Ribozyme-based capping assay on purified RNA (Agencourt AMPure XP purified). The results are summarized in Table 4.

TABLE 4
Effect of reduced spermidine concentrations
on RNA quality attributes

| | Spermidine | RNA yield | RNA Integrity | Capping |
|---|---|---|---|---|
| | [mM] | [g/L] | [%] | [%] |
| RNA R1 | 0 | 7.3 | 77.9 | 90.9 |
| | 0.5 | 7.2 | 78.3 | 90.3 |
| | 0.75 | 7.1 | 78 | 90.2 |
| | 1 | 6.9 | 77.2 | 90.1 |
| | 2 | 6.9 | 77.4 | 89.3 |
| RNA R3 | 0 | 6.0 | 74.7 | 90.1 |
| | 0.5 | 5.9 | 75.4 | 90.5 |
| | 0.75 | 5.9 | 74.4 | 90.6 |
| | 1 | 5.9 | 71.5 | 90.3 |
| | 2 | 5.6 | 71.6 | 89.8 |

### Result

As shown in Table 4, in the absence of spermidine, RNA yield and capping efficiency was improved. With increasing spermidine concentrations, the RNA yield and the capping efficiency was reduced. A negative effect of spermidine on RNA integrity was observed for RNA construct R3.

In summary, the reduction or the absence of spermidine had no negative effect on physiochemical properties on the RNA. Accordingly, RNA in vitro transcription using DNA beads should be performed with the lowest possible spermidine concentration (e.g. less than 2 mM or less than 1 mM) or in the absence of spermidine to avoid problems associated with DNA bead agglomeration.

### Example 8: Evaluation of Beneficial Molar Ratios of Mg²⁺ to NTP

As shown in previous Examples, MgCl₂ has an effect in inducing and/or stabilizing the agglomeration of DNA beads in an RNA in vitro transcription reaction. That effect is potentially caused by free Mg²⁺ anions in the IVT transcription composition that may cause bead aggregation and—as a consequence—reduces RNA yield. To evaluate the effect of Mg²⁺ even further, IVT reactions were performed to produce RNA R3 in the presence of varying concentrations of MgCl₂, NTPs, and spermidine.

PCR amplified biotinylated linear template DNA was immobilized on streptavidin functionalized magnetic beads (Dynabeads® M-450 E; Thermo Fisher) to produce DNA beads. The obtained magnetic DNA beads were used in a cycled RNA vitro transcription process in reaction tubes in the presence of varying spermidine concentrations.

For the RNA in vitro transcription of the present experiment, the following RNA in vitro transcription composition was used: 34 mg/mL DNA beads (60 ng/μL DNA), 80 mM Tris pH 8.0, 1 mM DTT, wherein the spermidine concentration, the NTPs and the MgCl₂ concentration was varied (see Table 5, Conditions A to E). Additionally, the RNA in vitro transcription composition comprised T7 RNA polymerase, Cap analog (CleanCap AG), pyrophosphatase and RNAse inhibitor in typical concentrations.

The IVT reactions were performed in 1.5 mL reaction tubes and incubated for about 90 min at 37° C. Two IVT cycles were preformed and the RNA yield was determined (using a Qubit BR RNA assay). The relative RNA yield (in comparison to 0 mM spermidine) was calculated. The results are summarized in Table 5.

TABLE 5
Effect of Mg2+ to NTP ratios in RNA in vitro transcription for the second IVT cycle

| | A | B | C | D | E |
|---|---|---|---|---|---|
| | 19 mM MgCl₂ | 14.2 mM MgCl₂ | 9.5 mM MgCl₂ | 19 mM MgCl₂ | 19 mM MgCl₂ |
| Spermidine | 27 mM NTP | 20.3 mM NTP | 13.5 mM NTP | 20.3 mM NTP | 13.5 mM NTP |
| [mM] | Mg²⁺/NTP: 0.7 | Mg²⁺/NTP: 0.7 | Mg²⁺/NTP: 0.7 | Mg²⁺/NTP: 0.94 | Mg²⁺/NTP: 1.4 |
| 0 | 100 | 100 | 100 | 100 | 100 |
| 1 | 83 | 78 | 74 | 92 | 56 |
| 2 | 86 | 106 | 80 | 90 | 11 |
| 3 | 66 | 88 | 66 | 64 | 7 |
| 4 | 85 | 110 | 88 | 37 | 3 |

### Result

As shown in Table 5, the highest RNA yield was typically achieved with 0 mM spermidine. Interestingly, the RNA yield dropped dramatically with increasing spermidine concentrations when the Mg²⁺/NTP was above 1.0 (see condition E where the relative RNA yield dropped from 100 to 11 in the presence of 2 mM spermidine). The RNA yield could be stabilized against the yield reduction induced spermidine by setting the Mg²⁺/NTP ratio to a value of below 1.4 (see condition D where the relative RNA yield dropped from 100 to 90 in the presence of 1 mM spermidine). The RNA yield could be further stabilized by setting the Mg²⁺/NTP ratio to a value of about 0.7 (see conditions A to C), even in the presence of larger amounts of spermidine.

In summary, RNA in vitro transcription yields using DNA beads were significantly improved by using a molar ratio of Mg²⁺ and NTP of below 1.4. Thereby, the levels of free Mg²⁺ in the reaction are reduced by e.g. complex formation with NTPs which reduces or prevents DNA bead agglomeration and hence a reduction of RNA yield. Moreover, the overall concentration of Mg²⁺ should be reduced (e.g. below 25 mM) to avoid unwanted DNA beads agglomeration.

### Example 9: Antioxidant Compounds have a Negative Impact on DNA Beads

In a stability study it has been observed that DNA beads as used herein (Dynabeads® M-450 E; Thermo Fisher) darken during the course of RNA in vitro transcription. As negative effects on the RNA yield and/or RNA quality can not be excluded (in particular not in the production of a therapeutic RNA) a route cause analysis was started that identified the antioxidant agent DTT as a potential cause.

Accordingly, DNA beads as used herein were incubated in IVT buffer (80 mM Tris pH 8.0, 2 mM spermidine) at increasing concentrations of DTT (0 mM to 40 mM) and stored for 65 h at 37° C. The color was visually inspected and compared to a control sample that was incubated in 1×TE buffer. The darkening of the beads was recorded and visually assessed at a scale from 0 (same color as control, red/orange) to 10 (black). The results are provided in Table 6. Moreover, test IVT reactions were carried out to evaluate the effect of reduced DTT on the RNA in vitro transcription yield (see Table 7).

TABLE 6
DTT has a negative impact on the physiochemical properties of DNA beads

| DTT concentration | 1 × TE | 0 mM | 1 mM | 2 mM | 5 mM | 10 mM | 20 mM | 40 mM |
|---|---|---|---|---|---|---|---|---|
| Color change | 0 | 0 | 1 | 1 | 3 | 4 | 6 | 8 |

TABLE 7
DTT has a negative impact on the physiochemical properties
of DNA beads when the IVT mix is incubated for 48 h
at 25° C. prior to transcription with DNA beads

| DTT concentration [mM] | RNA concentration [ng/μl] |
|---|---|
| 0 | 5555 |
| 1 | 7445 |
| 5 | 7738 |
| 40 | 7410 |

### Result

As shown in Table 6, the concentration of DTT had an impact on the physiochemical properties of the beads as the color started to darken at DTT concentrations above 1 mM. At the highest tested concentration (40 mM) the beads had a dark color. As shown in Table 7, the complete absence of DTT in the RNA in vitro transcription composition led to a lower RNA yield (RNA yield of 5555 ng/μl) as compared to the reactions comprising DTT (RNA yields of about 7500 ng/μl for 1 mM DTT to 40 mM DTT).

Overall, the data shows that, when performing RNA in vitro transcription using DNA beads (e.g. Dynabeads), the DTT concentration of the reaction should be reduced to avoid integrity problems of the beads. However, as DTT increases the RNA yield, the concentration of DTT should ideally be set to at least 1 mM.

1. An RNA in vitro transcription composition comprising DNA templates immobilized on beads contained in an RNA in vitro transcription (IVT) buffer, wherein the IVT buffer comprises
less than 2 mM spermidine; and
Mg²⁺ and NTP in a molar ratio of below 1.4.

2. The RNA in vitro transcription composition of claim 1, wherein the IVT buffer is configured to reduce or prevent agglomeration of the DNA beads during an RNA in vitro transcription.

3. The RNA in vitro transcription composition of claim 1 or 2, wherein the IVT buffer comprises Mg²⁺ at a concentration of less than about 25 mM.

4. The RNA in vitro transcription composition of claims 1 to 3, wherein the IVT buffer comprises Mg²⁺ at a concentration ranging from 2 mM to 20 mM.

5. The RNA in vitro transcription composition of claims 1 to 4, wherein the IVT buffer comprises less than 1.5 mM spermidine, preferably less than 1 mM spermidine.

6. The RNA in vitro transcription composition of claims 1 to 5, wherein the IVT buffer is essentially free of spermidine.

7. The RNA in vitro transcription composition of claims 1 to 6, wherein the IVT buffer comprises NTPs at a total concentration of at least about 3 mM.

8. The RNA in vitro transcription composition of claims 1 to 7, wherein the IVT buffer comprises NTPs at a total concentration ranging from 3 mM to 35 mM.

9. The RNA in vitro transcription composition of claim 7 or 8, wherein the NTPs comprise modified nucleotides, preferably selected from pseudouridine (ψ) or N1-methylpseudouridine (m1ψ).

10. The RNA in vitro transcription composition of claims 1 to 9, wherein the molar ratio of Mg²⁺ to NTP in the IVT buffer is below 1.2, preferably below 1.0.

11. The RNA in vitro transcription composition of claims 1 to 10, wherein the molar ratio of Mg²⁺ to NTP in the IVT buffer is ranging from 1.2 to 0.5, 1.0 to 0.5, preferably ranging from 0.8 to 0.6.

12. The RNA in vitro transcription composition of any one of the preceding claims, wherein the IVT buffer comprise a cap analogue, preferably a cap1 analogue.

13. The RNA in vitro transcription composition of any one of the preceding claims, wherein the IVT buffer comprises an antioxidant at a concentration of less than 5 mM, preferably at 1 mM.

14. The RNA in vitro transcription composition of any one of the preceding claims, wherein the IVT buffer comprises at least 5 units/ml RNA polymerase, optionally wherein the RNA polymerase is a T7 RNA polymerase.

15. The RNA in vitro transcription composition of any one of the preceding claims, wherein the composition comprises 10 μg/ml to 100 μg/ml immobilized DNA templates, preferably 50 μg/ml to 70 g/ml immobilized DNA templates.

16. The RNA in vitro transcription composition of any one of the preceding claims, wherein the composition is essentially free of non-immobilized DNA templates.

17. The RNA in vitro transcription composition of any one of the preceding claims, wherein the composition comprises 1 mg/ml to 100 mg/ml DNA beads, preferably 20 mg/ml to 40 mg/ml DNA beads.

18. The RNA in vitro transcription composition of any one of the preceding claims, wherein the bead surface comprises about 0.5 ng DNA/mm² to about 10 ng DNA/mm², preferably about 1 ng DNA/mm² to about 5 ng DNA/mm².

19. The RNA in vitro transcription composition of any one of the preceding claims, wherein the DNA beads have a diameter in a range of 1 μm to 10 μm, preferably in a range of 4 μm to 5 μm.

20. The RNA in vitro transcription composition of any one of the preceding claims, wherein the DNA beads comprise polysterene or a derivative of polysterene.

21. The RNA in vitro transcription composition of any one of the preceding claims, wherein the DNA beads are magnetic DNA beads, preferably (super) paramagnetic DNA beads.

22. The RNA in vitro transcription composition of any one of the preceding claims, wherein the DNA beads comprise a biotin-streptavidin immobilized DNA template.

23. The RNA in vitro transcription composition of any one of the preceding claims, wherein the DNA beads have been produced by site specific immobilization of a biotinylated PCR amplified DNA template on a streptavidin functionalized bead or by site specific immobilization of a biotinylated linearized plasmid DNA template on a streptavidin functionalized bead.

24. The RNA in vitro transcription composition of any one of the preceding claims, wherein the IVT buffer is configured to reduce or prevent DNA bead agglomeration at RNA concentrations of more than 100 mg/L.

25. The RNA in vitro transcription composition of any one of the preceding claims, wherein upon incubation of the RNA in vitro transcription composition for at least 1 h, less than 5% of the DNA beads contained in the composition are agglomerated.

26. Use of an RNA in vitro transcription composition of any one of the preceding claims in a manufacturing process of RNA.

27. A method for producing RNA comprising the steps of
(A) Providing an RNA in vitro transcription composition of any one of claims 1 to 25 comprising DNA beads in an IVT buffer contained in a reaction vessel;
(B) Performing an IVT cycle by mixing the DNA beads with the IVT buffer to produce an RNA batch.

28. The method of claim 27, wherein the method additionally comprises the step of
(C) Retaining the DNA beads in the reaction vessel and harvesting of the RNA batch

29. The method of claim 27 or 28, wherein the method additionally comprises at least one further IVT cycle that comprises the steps of
(E) Adding fresh IVT buffer into the reaction vessel that comprises the retained DNA beads;
(F) Performing of a further IVT cycle by mixing the DNA beads with the freshly added IVT buffer to produce a further RNA batch;
(G) Retaining the DNA beads in the reaction vessel and harvesting of the further RNA batch;

30. The method of claim 29, wherein the at least one further IVT cycle is repeated 1 to 20 times.

31. The method of claims 27 to 30, additionally comprising a step of
(D) Washing of the retained DNA beads in a wash buffer;
wherein step D is performed after step C and/or after step G.

32. The method of claim 31, wherein the wash buffer comprises at least one chelating agent.

33. The method of claim 32, wherein the chelating agent is EDTA or a derivative of EDTA.

34. The method of claims 31 to 33, wherein the wash buffer comprises urea.

35. The method of claims 31 to 34, wherein step D reduces or prevents agglomeration of the DNA beads and/or reduces or prevents attachment of RNA to the DNA beads.

36. The method of claims 27 to 35, additionally comprising at least one NTP feed step that is performed during the first IVT cycle (step B) and/or during a further IVT cycle (step F).

37. The method of claims 27 to 36, wherein the harvested RNA is essentially free of DNA beads or fragments thereof.

38. The method of claims 27 to 37, wherein the harvested RNA comprises RNA at a concentration of more than 500 mg/L, preferably at RNA concentrations of more than 1 g/L.

39. The method of claims 27 to 38, wherein the mixing of the DNA beads in steps B and/or F is performed by means of a magnet unit and/or wherein the retaining of the DNA beads in steps C and/or G is performed by means of a magnet unit.

40. The method of claims 27 to 39, wherein the providing step (A) comprises a DNA production step (A1), a DNA immobilization step (A2), and an optional on-bead DNA trimming step (A3).

41. The method of claims 27 to 40, additionally comprising a step (H) of purifying the harvested RNA.

42. The method of claim 41, wherein the RNA purification step (H) comprises at least one step selected from RP-HPLC, AEX, SEC, hydroxyapatite chromatography, TFF, filtration, precipitation, core-bead flow through chromatography, oligo(dT) purification, cellulose-based purification, or any combination thereof.

43. The method of claims 27 to 42, wherein the method does not comprise a DNAse treatment step.

44. The method of claims 27 to 43, wherein the method is for producing RNA that has a length of at least 1000 nucleotides.

45. The method of claims 27 to 44, wherein the method is for producing mRNA.

46. An RNA manufacturing device that comprises the RNA in vitro transcription composition of claims 1 to 25 and/or that is configured to perform the method of any one of the claims 27 to 45.

47. Use of an IVT buffer to reduce or prevent DNA bead agglomeration in an RNA manufacturing process, wherein the IVT buffer comprises less than 2 mM spermidine and Mg²⁺ to NTP in a molar ratio of not more than 1.4, and, optionally, less than 25 mM Mg²⁺

48. The use of claim 47, wherein the IVT buffer is further characterized by any of the features of claims 2 to 14 and, optionally, wherein the DNA beads are characterized by any of the features of claims 18 to 23.

49. Use of a wash buffer comprising a chelating agent to prevent or reduce agglomeration of DNA beads in an RNA manufacturing process, wherein the wash buffer comprises at least one chelating agent preferably at a concentration ranging from 1 mM to 10 mM.
